(19) United States
(12) Patent Application Publication
(10) Pub. No.: US 2025/0369157 A1
(43) Pub. Date: Dec. 4, 2025

(54) COMPOSITIONS AND METHODS FOR DNA LIBRARY PREPARATION

(71) Applicant: ILLUMINA, INC., San Diego, CA (US)

(72) Inventors: Sébastien Ricoult, Sawston (GB); Natalie Morrell, Cambridge (GB); Johan Basuki, Singapore (SG); Niall Gormley, Cambridge (GB); Claudia Lee, Singapore (SG)

(21) Appl. No.: 18/875,217

(22) PCT Filed: Mar. 22, 2024
(86) PCT No.: PCT/US2024/021089

§ 371 Date: Dec. 16, 2024

Related U.S. Application Data
(60) Provisional application No. 63/492,109, filed on Mar. 24, 2023.

(51) Int. Cl. C40B 50/06; C12Q 1/48; C12Q 1/6855; C12Q 1/686; C12Q 1/6869

(52) U.S. Cl. CPC C40B 50/06; C12Q 1/48; C12Q 1/6855; C12Q 1/686; C12Q 1/6869; C12Y 207/07

(57) ABSTRACT

The present disclosure relates, in general, to compositions for next generation sequencing and DNA library preparation, and systems and methods for use of the compositions in generating DNA libraries.

### CROSS REFERENCE TO RELATED APPLICATIONS

The present application claims the priority benefit of U.S. Provisional Patent Application No. 63/492,109, filed Mar. 24, 2023, incorporated by reference herein in its entirety.

### FIELD

The present application relates, in general, to compositions for next generation sequencing, DNA library preparation, and systems and methods for use of the compositions in generating DNA library preparations.

### BACKGROUND

Current protocols for DNA library preparation and next generation sequencing technology frequently employ a sample preparation process that converts DNA or RNA into a library of fragmented, sequenceable templates.

Libraries including polynucleotides are generally prepared in any suitable manner to attach oligonucleotide adapters to target polynucleotides. Sequencing may result in determination of the sequence of the whole, or a part of the target polynucleotides. The number of steps involved to transform nucleic acids into adapter-modified templates in solution ready for cluster formation on a substrate and sequencing can be reduced, or in some instances even minimized, by the use of transposase mediated fragmentation and tagging. This process, referred to as “tagmentation,” involves the fragmentation and adaptor tagging of nucleic acids by a transposome complex comprising transposase enzyme complexed with adapters comprising transposon end sequence, as described in, for example, US20180245069.

Sample preparation methods often involve multiple steps, material transfers, and expensive instruments to effect DNA fragmentation and DNA indexing, and, therefore, can be time-consuming and inefficient.

### SUMMARY

The present disclosure provides benefits and advantages to previous workflows for next generation sequencing by reducing the number of steps needed to proceed through the library preparation workflow. The present disclosure provides compositions comprising a series of lyophilized substances, e.g., microspheres or beads, containing library preparation reagents that release their contents upon predetermined release conditions such that the DNA preparation reactions can take place in a single container or in a minimal number of containers, thereby minimizing loss of DNA during addition or removal of reagents and transfer of samples to different sample containers. The lyophilization of the reagents and ability to combine multiple lyophilized microspheres into a single container provides for improved storage and stability of the reagents and leads to fewer handling steps and improved user experience.

Provided herein is a composition comprising:
a first lyophilized substance (e.g., microsphere, bead or cake) comprising one or more tagmentation reagents,
a plurality of first particles comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and
a plurality of second particles comprising a second shell surrounding a second core, wherein said second shell comprises a hydrophilic or hydrophobic polymer and has a second trigger release condition, and wherein said second core comprises one or more third lyophilized microspheres comprising one or more reagents for extension-ligation and PCR.

Also provided herein is a composition comprising:
a first lyophilized substance comprising one or more tagmentation reagents, and
a plurality of first particles comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and a second shell within the first core and surrounding a second core, wherein said second shell comprises a hydrophilic or hydrophobic polymer and has a second trigger release condition, and wherein said second core comprises one or more third lyophilized microspheres comprising one or more reagents for extension-ligation and PCR.

Also contemplated herein is a composition comprising:
a first lyophilized substance comprising one or more tagmentation reagents,
a plurality of first particles comprising a first shell surrounding a first core, wherein said first shell comprises a hydrophilic or hydrophobic polymer and has a first trigger release condition, and wherein said first core comprises one or more non-lyophilized microspheres or beads comprising sugar or plastic comprising one or more tagmentation poisoning reagents, optionally wherein one or more tagmentation poisoning reagents is coated and dried on the surface of the non-lyophilized microspheres or beads, and
a plurality of second particles comprising a second shell surrounding a second core, wherein said second shell comprises a hydrophilic or hydrophobic polymer and has a second trigger release condition, and wherein said second core comprises one or more non-lyophilized microspheres or beads comprising sugar or plastic comprising one or more extension-ligation and PCR reagents, optionally wherein one or more extension-ligation and PCR reagents is coated and dried on the surface of the non-lyophilized microspheres or beads.

Also provided herein is a composition comprising:
a first lyophilized substance comprising one or more tagmentation reagents, and
a plurality of first particles comprising a first shell surrounding a first core, wherein said first shell comprises a hydrophilic or hydrophobic polymer and has a first trigger release condition, and wherein said first core comprises one or more non-lyophilized microspheres or beads comprising sugar or plastic comprising one or more tagmentation poisoning reagents, optionally wherein one or more tagmentation poisoning reagents is coated and dried on the surface of the non-lyophilized microspheres or beads, and a second shell within the first core and surrounding a second core, wherein said second shell comprises a hydrophilic or hydrophobic polymer and has a second trigger release condition, and wherein said second core comprises one or more non-lyophilized microspheres or beads comprising sugar or plastic comprising one or more extension-ligation and PCR reagents, optionally wherein one or more extension-ligation and PCR reagents is coated and dried on the surface of the non-lyophilized microspheres or beads.

Provided herein is a composition comprising:
a first lyophilized substance (e.g., microsphere, bead or cake) comprising one or more tagmentation reagents,
a plurality of first particles comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a plurality of second particles comprising a second shell surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

Also provided herein is a composition comprising:
a first lyophilized substance comprising one or more tagmentation reagents, and
a plurality of first particles comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a second shell within the first core and surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

Also contemplated herein is a composition comprising:
a first lyophilized substance comprising one or more tagmentation reagents,
a plurality of first particles comprising a first inner shell surrounding a first core, wherein said first shell comprises a hydrophilic or hydrophobic polymer and has a first trigger release condition, and wherein said first core comprises one or more non-lyophilized microspheres or beads comprising sugar or plastic comprising one or more tagmentation poisoning reagents, optionally wherein one or more tagmentation poisoning reagents is coated and dried on the surface of the non-lyophilized microspheres or beads, and a first outer shell surrounding the first inner shell, wherein the first outer shell comprises a hydrophilic polymer and said first outer shell has a second trigger release mechanism; and
a plurality of second particles comprising a second shell surrounding a second core, wherein said second shell comprises a hydrophilic or hydrophobic polymer and has a third trigger release condition, and wherein said second core comprises one or more non-lyophilized microspheres or beads comprising sugar or plastic comprising one or more extension-ligation and PCR reagents, optionally wherein one or more extension-ligation and PCR reagents is coated and dried on the surface of the non-lyophilized microspheres or beads.

Also provided herein is a composition comprising:
a first lyophilized substance comprising one or more tagmentation reagents, and
a plurality of first particles comprising a first inner shell surrounding a first core, wherein said first inner shell comprises a hydrophilic or hydrophobic polymer and has a first trigger release condition, and wherein said first core comprises one or more non-lyophilized microspheres or beads comprising sugar or plastic comprising one or more tagmentation poisoning reagents, optionally wherein one or more tagmentation poisoning reagents is coated and dried on the surface of the non-lyophilized microspheres or beads, and a first outer shell surrounding the first inner shell, wherein the first outer shell comprises a hydrophilic polymer and said first outer shell has a second trigger release mechanism; and
a second shell within the first core and surrounding a second core, wherein said second shell comprises a hydrophilic or hydrophobic polymer and has a third trigger release condition, and wherein said second core comprises one or more non-lyophilized microspheres or beads comprising sugar or plastic comprising one or more extension-ligation and PCR reagents, optionally wherein one or more extension-ligation and PCR reagents is coated and dried on the surface of the non-lyophilized microspheres or beads.

In various implementations, the lyophilized substance is a lyophilized microsphere, lyophilized bead or lyophilized cake. In various implementations, the lyophilized cake comprises multiple stacked lyophilized cakes.

In various implementations, the polymer is a hydrophilic or water soluble polymer. In various implementations, the polymer is a hydrophobic or water insoluble polymer. In various implementations, the water insoluble polymer undergoes transition, either from hydrophobic to hydrophilic or from solid to liquid, upon trigger by the trigger release condition.

In various implementations, the first trigger release condition and second trigger release condition are different.

In various implementations, the first, second and/or third trigger release conditions are different. In various implementations, the first, second and/or third trigger release conditions are the same.

In various implementations, the trigger release condition is a time release condition, a temperature release condition or a pH release condition.

In various implementations, the one or more tagmentation reagents is a bead linked transposome (BLT), transposase, primers, buffers, divalent cations, magnesium sulphate, Tris buffer, cobalt buffer, and/or a lyophilization reagent.

In various implementations, the tagmentation reagents comprise bead linked transposomes, primer tags, cobalt chloride and trehalose. In various implementations, the tagmentation reagents comprise bead linked transposomes, magnesium sulfate, Tris buffer and trehalose

In various implementations, the one or more tagmentation poisoning reagents is a chelator, deoxynucleotide triphosphates (dNTPs), sodium dodecyl sulfate (SDS), Tris buffer, potassium buffer, ammonium salts, and/or a lyophilization reagent.

In various implementations, the tagmentation poisoning reagents comprise Tris buffer, potassium chloride, ammonium sulfate, cyclen tetrahydrochloride, alpha cyclodextrin, dNTPs and trehalose. In various implementations, the tagmentation poisoning reagents comprise potassium chloride, ammonium sulfate, SDS, dNTPs and trehalose.

In various implementations, the extension-ligation and PCR reagents comprise alpha cyclodextrin, magnesium sulfate, one or more polymerases, ampligase, NAD+, betaine, a surfactant and/or a lyophilization reagent.

In various implementations, the extension-ligation and PCR reagents comprise magnesium sulfate, glycerol free hot start Q5, tTaq 608 polymerase, ampligase, NAD+, betaine, Tween-20 and trehalose. In various implementations, the extension-ligation and PCR reagents comprise glycerol free hot start Q5, tTaq 608 polymerase, ampligase, NAD+, betaine, alpha cyclodextrin, Tween-20 and trehalose.

In various implementations, the first and/or second shell comprises temperature-triggered release polymers and/or time-delayed release polymers. In various implementations, the first and/or second shell comprises temperature-triggered release polymers, time-delayed release polymers and/or functional additives. In various implementations, the first and/or second shell comprises an anti-static agent, an anti-foaming agent, a plasticizer, one or more upper critical solution temperature (UCST) polymers, one or more lower critical solution temperature (LCST) polymers, hydroxypropyl methylcellulose (HPMC), or combinations thereof.

In various implementations, the first and/or second shell comprises Efka 6783, Makon 17R4, magnesium stearate, UCST polymer 1, UCST polymer 2, LCST polymer, hydroxypropyl methylcellulose (HPMC), or combinations thereof. UCST polymer 1 can also be referred to herein as a first UCST polymer, and UCST polymer 2 can also be referred to herein as a second UCST polymer.

In various implementations, the first and/or second shell comprises Efka 6783, Makon 17R4, polyethylene glycol (PEG) stearate, UCST polymer 1, UCST polymer 2, LCST polymer, hydroxypropyl methylcellulose (HPMC), or combinations thereof.

In various implementations, the first shell comprises a UCST/LCST polymer and one or more functional additives, such as anti-tacking agent, anti-foaming agent, plasticizer, anti-static agent or combinations thereof. In various implementations, the first shell comprises Efka 6783, Makon 17R4, magnesium stearate, and a UCST polymer 1.

In various implementations, the first shell comprises a UCST/LCST polymer and one or more functional additives, such as anti-tacking agent, anti-foaming agent, plasticizer, anti-static agent or combinations thereof. In various implementations, the first shell comprises Efka 6783, Makon 17R4, PEG stearate, and a UCST polymer 1.

In various implementations, when the particle comprises a first inner shell and a first outer shell, the first inner shell comprises a hydrophobic polymer. In various embodiments, the first inner shell comprises cellulose acetate (CA), cellulose acetate phthalate (CAP), cellulose acetate trimellitate (CAT), ethyl cellulose (EC), methylethyl cellulose (MEC), polyvinyl acetate, fatty acid, fatty acid esters, or combinations thereof. In various embodiments, the first inner shell comprises cellulose acetate.

In various implementations, when the particle comprises a first inner shell and a first outer shell, the first outer shell comprises a hydrophilic polymer. In various embodiments, the first outer shell comprises carboxymethyl cellulose (CMC), carboxymethyl ethylcellulose (CMEC), hydroxyethyl cellulose (HEC), hydroxypropyl cellulose (HPC), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), ethylhydroxy ethylcellulose (EHEC), pullulan, polyvinyl pyrrolidone, polyvinyl alcohol, copolymers of vinyl pyrrolidone or vinyl alcohol, polyacrylamide, polyacrylic acid, copolymers of ethacrylic acid or methacrylic acid and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride, P(NIPAAm-co-nBu) LCST15° C. or combinations thereof. In various implementations, the first outer shell comprises hydroxypropyl methyl cellulose (HPMC) or P(NIPAAm-co-nBu) LCST15° C. In various implementations, the first outer shell comprises hydroxypropyl methyl cellulose (HPMC)

In various implementations, the second shell comprises hydroxypropyl methylcellulose (HPMC), UCST/LCST polymer and one or more functional additives, such as anti-tacking agent, anti-foaming agent, plasticizer, anti-static agent or combinations thereof. In various implementations, the second shell comprises Efka 6783, Makon 17R4, magnesium stearate, a UCST polymer 2 and hydroxypropyl methylcellulose (HPMC).

In various implementations, the first release condition is a temperature release condition. In various implementations, the temperature for the first release condition is above about 45° C. Exemplary temperature-triggered release polymers are UCST polymers, e.g., P (acrylonitrile-co-acrylamide), P(N-acryloylglycinamide-co-styrene), and LCST polymers, e.g., P(N-isopropylacrylamide) derivatives.

In various implementations, the second release condition is a time release condition. Exemplary time-delayed release polymers are hydroxypropyl methylcellulose (HPMC), Poly(vinylpyrrolidone-co-vinyl acetate) and cellulose acetate.

In various implementations, the first or second release condition is a pH-dependent release condition. Exemplary pH-dependent release polymers include polymers that dissolve at different pH levels, e.g., from between about pH 4.5 to about pH 9, or from about pH 6 to about pH 8.5. Exemplary polymers include, but are not limited to polymers that dissolve at or below pH 5: cellulose acetate, timellitate, cellulose acetate phthalate, PVA phthalate, HPMC phthalate; chitosan (soluble below pH 6); polymers that dissolve between pH 5 and 6 or above: Kollicoat MAE 100, Kollicoat 30D, Eudragit L100, Eudragit L100-55, Eudragit L30D-55, Eudragit L12.5, polymers that dissolve between about pH 6 and about pH 7: Eudragit L100/S100 1:1, Eudragit L/S12.5, Eudragit S100, Eudragit FS30D, Eudragit S12.5, shellac; carboxy methylcellulose (soluble above pH 7.4), or rennet casein (soluble around pH 9).

In various implementations, the composition is stable when stored between about 4° C. to about 37° C.

The disclosure also contemplates one or more containers for preparing a DNA library comprising a composition as described herein.

In various implementations, the container comprises:
a first lyophilized substance comprising one or more tagmentation reagents,
a plurality of first particles comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and
a plurality of second particles comprising a second shell surrounding a second core, wherein said second shell comprises a hydrophilic or hydrophobic polymer and has a second trigger release condition, and wherein said second core comprises one or more third lyophilized microspheres comprising one or more extension-ligation and PCR reagents.

In various implementations, the container comprises:
a first lyophilized substance comprising one or more tagmentation reagents, and
a plurality of first particles comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents.

In various implementations, the container comprises:
a plurality of second particles comprising a second shell surrounding a second core, wherein said second shell comprises a hydrophilic or hydrophobic polymer and has a second trigger release condition, and wherein said second core comprises one or more third lyophilized microspheres comprising one or more extension-ligation and PCR reagents.

In various implementations, the container comprises:
a first lyophilized substance comprising one or more tagmentation reagents,
a plurality of first particles comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a plurality of second particles comprising a second shell surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

In various implementations, the container comprises:
a first lyophilized substance comprising one or more tagmentation reagents, and
a plurality of first particles comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a second shell within the first core and surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

In various implementations, the container comprises:
a first lyophilized substance comprising one or more tagmentation reagents, and
a plurality of first particles comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents and a first outer shell surrounding the first inner shell, wherein the first outer shell comprises a hydrophilic polymer and said first outer shell has a second trigger release mechanism.

In various implementations, the container comprises:
a plurality of second particles comprising a second shell surrounding a second core, wherein said second shell comprises a hydrophilic or hydrophobic polymer and has a third trigger release condition, and wherein said second core comprises one or more third lyophilized microspheres comprising one or more extension-ligation and PCR reagents.

In various implementations, the container is a PCR tube, vial, microtube, flow cell, multiwell plate, e.g., a 96- or 384-well plate, glass tube, transwell membrane/mesh insert, cartridge or microfluidic chip.

Also provided is a kit comprising one or more compositions or containers as described herein and instructions for use. In one implementation a kit comprises a container comprising a first lyophilized substance and a first particle and a separate container comprising a plurality of second particles.

The disclosure contemplates a method of preparing a DNA library comprising:
adding a sample comprising DNA to a container comprising a composition as described herein, wherein addition of the sample initiates dissolution of the first lyophilized substance to release one or more tagmentation reagents into the container to initiate a tagmentation reaction;
allowing the tagmentation reaction to continue under conditions and for a sufficient period to complete tagmentation of the DNA in the sample;
initiating the first trigger release condition of the first shell, wherein the first trigger release condition of the first shell releases one or more tagmentation poisoning reagents of the first core into the container to initiate a tagmentation poisoning reaction; and
allowing the tagmentation poisoning reaction to continue under conditions and for a sufficient period to initiate the second trigger release condition of the second shell, wherein one or more extension-ligation and PCR reagents are released from the second core, wherein the extension-ligation and PCR reagents carry out gap fill and PCR amplification.

Also provided is a method of preparing a DNA library comprising:
adding a sample comprising DNA to a container comprising a composition as described herein, wherein addition of the sample initiates dissolution of the first lyophilized substance to release one or more tagmentation reagents into the container to initiate a tagmentation reaction;
allowing the tagmentation reaction to continue under conditions and for a sufficient period to at least substantially complete tagmentation of the DNA in the sample;
initiating the second trigger release condition of the first outer shell, which subsequently initiates the first trigger release condition for the first inner shell, wherein the first trigger release condition of the first inner shell releases one or more tagmentation poisoning reagents of the first core into the container to initiate a tagmentation poisoning reaction; and,
allowing the tagmentation poisoning reaction to continue under conditions and for a sufficient period to initiate the second trigger release condition of the second shell, wherein one or more extension-ligation and PCR reagents are released from the second core, wherein the extension-ligation and PCR reagents carry out gap fill and PCR amplification.

In various implementations, the method further comprises isolating the DNA and sequencing the DNA library.

The disclosure also contemplates a method of preparing a DNA library comprising:
adding a sample comprising DNA to a system as described herein, wherein addition of the sample initiates dissolution of the first lyophilized substance to release one or more tagmentation reagents into the container to initiate a tagmentation reaction;
allowing the tagmentation reaction to continue under conditions and for a sufficient period to complete tagmentation of the DNA in the sample;
initiating the first trigger release condition of the first shell, wherein the first trigger release condition of the first shell releases one or more tagmentation poisoning reagents of the first core into the container to initiate a tagmentation poisoning reaction; and
allowing the tagmentation poisoning reaction to continue under conditions and for a sufficient period to initiate the second trigger release condition of the second shell, wherein one or more extension-ligation and PCR reagents are released from the second core, wherein the extension-ligation and PCR reagents carry out gap fill and PCR amplification.

In various implementations, the method further comprises isolating the DNA and sequencing the DNA library.

In various implementations, the tagmentation reaction is carried out between about 37° C. to about 41° C. for about 3 minutes to about 15 minutes, or about 5 minutes to about 10 minutes. In various implementations, the tagmentation reaction is carried out between about 37° C. to about 41° C. for about 5 minutes.

In various implementations, the first release condition is a temperature release condition. In various implementations, the temperature for the first temperature release condition is above about 45° C.

In various implementations, the tagmentation poisoning reaction is carried out at between about 55° C. to about 65° C. for about 1 to about 5 minutes. In various implementations, the tagmentation poisoning reaction is carried out at between about 60° C. to about 65° C. for about 1 to about 5 minutes.

In various implementations, the second release condition is a time release condition. In various implementations, the time release condition is from about 1 to about 3 minutes at about 50° C.

In various implementations, the first, second or third release condition is a pH dependent release condition. In various implementations, the pH-dependent release condition is from about pH 4.5 to about pH 9 or from about pH 6 to about pH 8.5. Exemplary pH-dependent release polymers include polymers that dissolve at or below pH 5: cellulose acetate, timellitate, cellulose acetate phthalate, PVA phthalate, HPMC phthalate; chitosan (soluble below pH 6); polymers that dissolve between pH 5 and 6 or above: Kollicoat MAE 100, Kollicoat 30D, Eudragit L100, Eudragit L100-55, Eudragit L30D-55, Eudragit L12.5, polymers that dissolve between about pH 6 and about pH 7: Eudragit L100/S100 1:1, Eudragit L/S12.5, Eudragit S100, Eudragit FS30D, Eudragit S12.5, shellac; carboxy methylcellulose (soluble above pH 7.4), or rennet casein (soluble around pH 9).

In various implementations, the one or more extension-ligation and PCR reagents are released from the second core at about 50° C., wherein the extension-ligation and PCR reagents carry out extension, ligation gap fill and PCR amplification.

It is understood that each feature or implementation, or combination, described herein is a non-limiting, illustrative example of any of the aspects of the invention and, as such, is meant to be combinable with any other feature or implementation, or combination, described herein. For example, where features are described with language such as “one implementation”, “various implementations”, “some implementations”, “certain implementations”, “further implementation”, “specific exemplary implementations”, and/or “another implementation”, each of these types of implementations is a non-limiting example of a feature that is intended to be combined with any other feature, or combination of features, described herein without having to list every possible combination.

Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 shows a comparison of DNA yield after tagmentation reaction using either liquid or lyophilized buffers.

FIG. 2 shows a comparison of DNA yield after the tagmentation poisoning reaction using either liquid or lyophilized buffers.

FIG. 3 shows a comparison of DNA yield after the PCR reaction using either liquid or lyophilized buffers.

FIG. 4 depicts optional workflows for completing initial steps in DNA library preparation using lyophilized microsphere compositions.

FIG. 5 shows the amount of DNA generated from a PCR reaction when lyophilized microspheres comprising magnesium were encapsulated in wax.

FIG. 6 shows a workflow for DNA library preparation according to one implementation of the disclosure.

### DETAILED DESCRIPTION

This disclosure relates to an improved workflow for preparing DNA libraries by reducing the number of interactive steps, or touchpoints, involved in the process of DNA library preparation, e.g., from tagmentation, Tn5 removal, Gap-fill and PCR amplification. The workflow comprises use of a series of multiple lyophilized microspheres comprising the reagents used to complete DNA library preparation. Use of the multiple microspheres encapsulating the different sequence of reagents allows for the majority of workflow steps to be completed in a single container, minimizing sample loss during handling. Additionally, the use of lyophilized microspheres reduces the liquid volume of the reagents used allowing for an increased volume of sample to be used per reaction.

The compositions, systems and methods described herein for DNA library preparation may also be used within a workflow for other methods of DNA library preparation, such as spatial indexing, sequencing by synthesis (SBS) and other next-generation sequencing (NGS) technologies.

It is to be appreciated that certain aspects, modes, implementations, variations, and features of the present disclosure are described below in various levels of detail in order to provide a substantial understanding of the present technology. Unless otherwise noted, all technical and scientific terms used herein generally have the same meaning as commonly understood by one of ordinary skill in the art. The use of the term “including” as well as other forms is not limiting. The use of the term “having” as well as other forms is not limiting. As used in this disclosure, whether in a transitional phrase or in the body of the claim, the terms “comprise(s)” and “comprising” are to be interpreted as having an open-ended meaning. That is, the terms are to be interpreted synonymously with the phrases “having at least” or “including at least.”

The term “about,” particularly in reference to a given quantity, is meant to encompass deviations of plus or minus five percent.

As used herein, the terms “includes,” “including,” “includes,” “including,” “contains,” “containing,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter.

The terms “substantially”. “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “connect”, “contact”, and/or “coupled” include a variety of arrangements and assemblies. These arrangements and techniques include, but are not limited to, (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (optionally with the presence of one or more additional components therebetween). Components that are in direct physical contact with one another may or may not be in electrical contact and/or fluid contact with one another. Moreover, two components that are electrically connected, electrically coupled, optically connected, optically coupled, fluidly connected, or fluidly coupled may or may not be in direct physical contact, and one or more other components may be positioned between those two connected components.

As described herein, the terms “polynucleotide” or “nucleic acids” refer to deoxyribonucleic acid (DNA), ribonucleic acid (RNA), or analogs of either DNA or RNA made from nucleotide analogs. The terms as used herein also encompasses cDNA, that is complementary, or copy DNA produced from an RNA template, for example by the action of reverse transcriptase.

Nucleic acids include naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art such as peptide nucleic acid (PNA) or locked nucleic acid (LNA). Naturally occurring nucleic acids generally have a deoxyribose sugar (e.g. found in deoxyribonucleic acid (DNA)) or a ribose sugar (e.g. found in ribonucleic acid (RNA)).

A nucleic acid can contain any of a variety of analogs of these sugar moieties that are known in the art. A nucleic acid can include native or non-native bases. A native deoxyribonucleic acid can have one or more bases selected from the group consisting of adenine, thymine, cytosine, or guanine and a ribonucleic acid can have one or more bases selected from the group consisting of uracil, adenine, cytosine or guanine. Useful non-native bases that can be included in a nucleic acid are known in the art.

The term nucleotide as described herein may include natural nucleotides, analogs thereof, ribonucleotides, deoxyribonucleotides, dideoxyribonucleotides and other molecules known as nucleotides. As described herein, a nucleotide may include a nitrogen containing heterocyclic base, a sugar, and one or more phosphate groups. Nucleotides may be monomeric units of a nucleic acid sequence, for example to identify a subunit present in a DNA or RNA strand. A nucleotide may also include a molecule that is not necessarily present in a polymer, for example, a molecule that is capable of being incorporated into a polynucleotide in a template dependent manner by a polymerase. A nucleotide may include a nucleoside unit having, for example, 0, 1, 2, 3 or more phosphates on the 5′ carbon. Tetraphosphate nucleotides, pentaphosphate nucleotides, and hexaphosphate nucleotides may be useful, as may be nucleotides with more than 6 phosphates, such as 7, 8, 9, 10, or more phosphates, on the 5′ carbon. Examples of naturally occurring nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP.

Non-natural nucleotides include nucleotide analogs, such as those that are not present in a natural biological system or not substantially incorporated into polynucleotides by a polymerase in its natural milieu, for example, in a non-recombinant cell that expresses the polymerase. Non-natural nucleotides include those that are incorporated into a polynucleotide strand by a polymerase at a rate that is substantially faster or slower than the rate at which another nucleotide, such as a natural nucleotide that base-pairs with the same Watson-Crick complementary base, is incorporated into the strand by the polymerase. For example, a non-natural nucleotide may be incorporated at a rate that is at least 2 fold different, 5 fold different, 10 fold different, 25 fold different, 50 fold different, 100 fold different, 1000 fold different, 10000 fold different, or more when compared to the incorporation rate of a natural nucleotide. A non-natural nucleotide can be capable of being further extended after being incorporated into a polynucleotide. Examples include, nucleotide analogs having a 3′ hydroxyl or nucleotide analogs having a reversible terminator moiety at the 3′ position that can be removed to allow further extension of a polynucleotide that has incorporated the nucleotide analog. Examples of reversible terminator moieties are described, for example, in U.S. Pat. Nos. 7,427,673, 7,414,116, and 7,057,026, as well as WO 91/06678 and WO 07/123744, each of which is hereby incorporated by reference in its entirety. It will be understood that in some examples a nucleotide analog having a 3′ terminator moiety or lacking a 3′ hydroxyl (such as a dideoxynucleotide analog) can be used under conditions where the polynucleotide that has incorporated the nucleotide analog is not further extended. In some examples, nucleotide(s) may not include a reversible terminator moiety, or the nucleotides(s) will not include a non-reversible terminator moiety or the nucleotide(s) will not include any terminator moiety at all.

This disclosure encompasses nucleotides including a fluorescent label (or any other detection tag) that may be used in any method disclosed herein, on its own or incorporated into or associated with a larger molecular structure or conjugate.

The fluorescent label can include compounds selected from any known fluorescent species, for example rhodamines or cyanines. A fluorescent label as disclosed herein may be attached to any position on a nucleotide base, and may optionally include a linker. The function of the linker is generally to aid chemical attachment of the fluorescent label to the nucleotide. In particular implementations Watson-Crick base pairing can still be carried out for the resulting analogue. A linker group may be used to covalently attach a dye to the nucleoside or nucleotide. A linker moiety may be of sufficient length to connect a nucleotide to a compound such that the compound does not significantly interfere with the overall binding and recognition of the nucleotide by a nucleic acid replication enzyme. Thus, the linker can also include a spacer unit. The spacer distances, for example, the nucleotide base from a cleavage site or label.

As used herein, the term “substrate” is intended to mean a solid support or support structure. The term includes any material that can serve as a solid or semi-solid foundation for creation of features such as wells for the deposition of biopolymers, including nucleic acids, polypeptide and/or other polymers. A substrate as provided herein is modified, for example, or can be modified to accommodate attachment of biopolymers by a variety of methods well known to those skilled in the art. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, a variety of polymers other than those exemplified above and multiwell microtiter plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

In some implementations, the solid support comprises one or more surfaces that are accessible to contact with reagents, beads, or analytes. The surface can be substantially flat or planar. Alternatively, the surface can be rounded or contoured. Example contours that can be included on a surface are wells (e.g., microwells or nanowells), depressions, pillars, ridges, channels or the like. Example materials that can be used as a surface include glass such as modified or functionalized glass; plastic such as acrylic, polystyrene or a copolymer of styrene and another material, polypropylene, polyethylene, polybutylene, polyurethane or TEFLON; polysaccharides or cross-linked polysaccharides such as agarose or Sepharose; nylon; nitrocellulose; resin; silica or silica-based materials including silicon and modified silicon, carbon-fiber; metal; inorganic glass; optical fiber bundle, or a variety of other polymers. A single material or mixture of several different materials can form a surface useful in certain examples. In some examples, a surface comprises wells (e.g., microwells or nanowells). In some aspects, the surface comprises wells in an array of wells (e.g., microwells or nanowells) on glass, silicon, plastic or other suitable solid supports with patterned, covalently-linked gel such as poly(N-(5-azidoacetamidylpentyl) acrylamide-coacrylamide) (PAZAM, see, for example, U.S. Pat. App. Pub. No. 2014/0079923 A1, which is incorporated herein by reference). In some examples, a support structure can include one or more layers.

As used herein, “derivative” or “analogue” means a synthetic nucleotide or nucleoside derivative having modified base moieties and/or modified sugar moieties. Such derivatives and analogs are discussed in, for example, Bücher, NUCLEOTIDE ANALOGS (John Wiley & Son, 1980) and Uhlmann et al., “Antisense Oligonucleotides: A New Therapeutic Principle,” Chemical Reviews 90:543-584 (1990), both of which are hereby incorporated by reference in their entirety. Nucleotide analogs may also include modified phosphodiester linkages, including phosphorothioate, phosphorodithioate, alkyl-phosphonate, phosphoranilidate and phosphoramidate linkages. “Derivative”. “analog”, and “modified” as used herein, may be used interchangeably, and are encompassed by the terms “nucleotide” and “nucleoside” as described herein.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges. An exemplary number of features within a microarray includes a plurality of about 500,000 or more discrete features within 1.28 cm². Exemplary nucleic acid pluralities include, for example, populations of about 1×10⁵, 5×10⁵ and 1×10⁶ or more different nucleic acid species. Accordingly, the definition of the term is intended to include all integer values greater than two. An upper limit of a plurality can be set, for example, by the theoretical diversity of nucleotide sequences in a nucleic acid sample.

As used herein, the term “double-stranded,” when used in reference to a nucleic acid molecule, means that substantially all of the nucleotides in the nucleic acid molecule are hydrogen bonded to a complementary nucleotide. A partially double stranded nucleic acid can have at least 10%, 25%, 50%, 60%, 70%, 80%, 90% or 95% of its nucleotides hydrogen bonded to a complementary nucleotide.

As used herein, the term “single-stranded,” when used in reference to a nucleic acid molecule, means that essentially none of the nucleotides in the nucleic acid molecule are hydrogen bonded to a complementary nucleotide.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T,A:U, and G:C, by Watson-Crick and Hoogsteen-type hydrogen bonding. In certain implementations, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application, and can be routinely determined by persons skilled in the art, without undue experimentation.

As used herein, the term “hybridization” refers to the process in which two single-stranded polynucleotides bind non-covalently to form a stable double-stranded polynucleotide. A resulting double-stranded polynucleotide is a “hybrid” or “duplex.” Hybridization conditions will typically include salt concentrations of less than about 1 M, more usually less than about 500 mM and may be less than about 200 mM. A hybridization buffer includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to the other, uncomplimentary sequences. Stringent conditions are sequence-dependent and are different in different circumstances, and may be determined routinely by those skilled in the art.

As used herein, the term “dNTP” refers to deoxynucleoside triphosphates. The purine bases (Pu) include adenine (A), guanine (G) and derivatives and analogs thereof. The pyrimidine bases (Py) include cytosine (C), thymine (T), uracil (U) and derivatives and analogs thereof.

As used herein, the terms “ligation,” “ligating,” and grammatical equivalents thereof are intended to mean to form a covalent bond or linkage between the termini of two or more nucleic acids, e.g., oligonucleotides and/or polynucleotides. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide. The term “ligation” also encompasses non-enzymatic formation of phosphodiester bonds, as well as the formation of non-phosphodiester covalent bonds between the ends of oligonucleotides, such as phosphorothioate bonds, disulfide bonds, and the like.

As used herein, the term “each,” when used in reference to a collection of items, is intended to identify an individual item in the collection but does not necessarily refer to every item in the collection unless the context clearly dictates otherwise.

As used herein, the term “extend,” when used in reference to a nucleic acid, is intended to mean addition of at least one nucleotide or oligonucleotide to the nucleic acid. In particular implementations one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended.

As used herein, the term “tagmentation,” “tagment,” or “tagmenting” refers to transforming a nucleic acid, e.g., a DNA, into adaptor-modified templates in solution ready for cluster formation and sequencing by the use of transposase mediated fragmentation and tagging. This process often involves the fragmentation and adapter tagging of the nucleic acid by a transposome complex comprising transposase enzyme complexed with a recognition sequence and adaptors comprising transposon end sequence. Tagmentation results in the simultaneous fragmentation of the nucleic acid and appending of the adaptors to the 5′ ends of the double stranded DNA. Following removal of the transposase enzyme, additional sequences are added to the ends of the adapted fragments by extension-ligation and PCR.

A “transposase” means an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US Pat. Publ. No. 2010/0120098, the content of which is incorporated herein by reference in its entirety. Although implementations described herein refer to Tn5 transposase and/or hyperactive Tn5 transposase, it will be appreciated that any transposition system that is capable of inserting a transposon end with sufficient efficiency to 5′-tag and fragment a target nucleic acid for its intended purpose can be used in the present invention. In particular implementations, a preferred transposition system is capable of inserting the transposon end in a random or in an almost random manner to 5′-tag and fragment the target nucleic acid.

As used herein, the term “transposition reaction” refers to a reaction wherein one or more transposons are inserted into target nucleic acids, e.g., at random sites or almost random sites. Essential components in a transposition reaction are a transposase and DNA oligonucleotides (combined into the transposome prior to the tagmentation reaction) that exhibit the nucleotide sequences of a transposon, including the transferred transposon sequence and its complement (the non-transferred transposon end sequence) as well as other components needed to form a functional transposition or transposome complex. The DNA oligonucleotides can further comprise additional sequences (e.g., adaptor or primer sequences) as needed or desired. In some implementations, the method provided herein is exemplified by employing a transposition complex formed by a hyperactive Tn5 transposase and a Tn5-type transposon end (Goryshin and Reznikoff, 1998,J. Biol. Chem., 273:7367) or by a MuA transposase and a Mu transposon end comprising Rland R2 end sequences (Mizuuchi, 1983, Cell, 35:785; Savilahti et al., 1995, EMBO J., 14:4893). However, any transposition system that is capable of inserting a transposon end in a random or in an almost random manner with sufficient efficiency to 5′-tag and fragment a target DNA for its intended purpose can be used in the present invention.

Examples of transposition systems known in the art which can be used for the present methods include but are not limited to Staphylococcus aureus Tn552 (Colegio et al., 2001, J Bacterid., 183:2384-8; Kirby et al., 2002, Mol Microbiol, 43:173-86), Tyl(Devine and Boeke, 1994, NucleicAcids Res., 22:3765-72 and International Patent Application No. WO 95/23875), TransposonTn7 (Craig, 1996, Science. 271:1512; Craig, 1996, Review in: Curr Top MicrobiolImmunol, 204:27-48), TnIO and ISIO (Kleckner et al., 1996, Curr Top Microbiol Immunol,204:49-82), Mariner transposase (Lampe et al., 1996, EMBO J., 15:5470-9), Tci (Plasterk, 1996, Curr Top Microbiol Immunol, 204:125-43), P Element (Gloor, 2004, Methods Mol Biol, 260:97-114), TnJ (Ichikawa and Ohtsubo, 1990, J Biol Chem. 265:18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86:2525-9), and retrotransposon of yeast (Boeke and Corces, 1989, Annu Rev Microbiol. 43:403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein uses, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the invention include but are not limited to wild-type, derivative or mutant transposon end sequences that form a complex with a transposase chosen from among a wild-type, derivative or mutant form of the transposase.

As used herein, the term “transposome complex” refers to a transposase enzyme non-covalently bound to a double stranded nucleic acid. For example, the complex can be a transposase enzyme pre-incubated with double-stranded transposon DNA under conditions that support non-covalent complex formation. Double-stranded transposon DNA can include, without limitation, Tn5 DNA, a portion of Tn5 DNA (e.g., Tn5 recognition site), a transposon end composition, a mixture of transposon end compositions or other double-stranded DNAs capable of interacting with a transposase such as the hyperactive Tn5 transposase.

The compositions, systems, and methods described herein include particles having a shell surrounding a core and the core may include one or more lyophilized microspheres (i.e., the composition may include an encapsulated lyophilized microsphere) or a lyophilized substance.

As described herein, “encapsulate”, “encapsulated”, and “encapsulation” include the enclosing of one or more materials or substances, for example microspheres as described herein. Microencapsulation as described herein refers to the embedding of at least one ingredient, for example, an active agent, into at least one other material, for example, a shell material. Encapsulation in accordance with the present disclosure includes, but is not limited to, bulk encapsulation, macroencapsulation, microencapsulation, nano encapsulation, single molecule, and ionic encapsulation. In accordance with the present disclosure, the compositions, systems, and methods described herein have many benefits including, for example, increasing stability of microspheres, use of macroencapsulation to enable multi-run cartridges, and use of microencapsulation to enable simplified workflows and reduced number of reagent wells. The compositions, systems, and methods described herein use encapsulation of particles that would otherwise be responsive to pH changes to stabilize these buffers and increase SBS performance. The compositions, systems, and methods described herein also use encapsulation to reduce the risk of static charge that otherwise presents difficulty for dispensing and dry compounding microspheres during manufacturing.

As used herein, “microsphere” includes spherical particles that include a shell and a core and have a diameter of 0.1 μm to 1,000 μm. For example, a microsphere may have a diameter of about 0.1 μm, 0.5 μm, 1 μm, 10 μm, 20 μm, 30 μm, 40 μm, 50 μm, 60 μm, 70 μm, 80 μm, 90 μm, 100 μm, 150 μm, 200 μm, 300 μm, 400 μm, 500 μm, 600 μm, 700 μm, 800 μm, 900 μm, 1000 μm, or any diameter between about 0.1 μm and about 1,000 μm. In one implementation, the encapsulated microsphere has a diameter between about 100 μm and 1000 μm.

Microspheres may refer to lyophilized particles comprising reagents and/or active ingredients. In certain implementations, microspheres may comprise a polymer shell, for example, biodegradable polymers and/or water soluble polymers, and optionally an inner core inside the shell. Microspheres in accordance with the present disclosure include those prepared by conventional techniques, which are known to those skilled in the art. For example, microspheres may be prepared by freezing a liquid into frozen pellets, followed by placing frozen microspheres in a dryer, for example, a rotational dryer.

As described herein, a “shell” includes a composition that surrounds a core. In one implementation, a shell includes an outer layer of a microsphere and, or in the alternative, an outer layer of a macrosphere. In one implementation, the shell includes, for example, a shell material selected from the group consisting of carrageenan, agarose, poloxamer, shellac, trehalose, paraffin wax, fatty acid (myristic acid, palmitic acid), fatty acid ester (Span 60, PEG40 stearate), gelatin, hydroxypropyl methylcellulose (HPMC), cellulose acetate, pullalan, oxygen scavenger, alginate, chitosan, starch film, benzoxaborole-poly(vinyl alcohol) (benzoxaborole-PVA), pectin, polyvinylpyrrolidone (PVP), poly(vinylpyrrolidone-co-vinyl acetate), polyvinyl alcohol (PVA), Poly(vinylalcohol-graft-PEG), UCST polymers, LCST polymers, or any combination thereof.

Water soluble (hydrophilic) polymers include carboxymethyl cellulose (CMC), carboxymethyl ethylcellulose (CMEC), hydroxyethyl cellulose (HEC), hydroxypropyl cellulose (HPC), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC), pullulan, polyvinyl pyrrolidone, polyvinyl alcohol, copolymers of vinyl pyrrolidone or vinyl alcohol, polyacrylamide, polyacrylic acid, copolymers of ethacrylic acid or methacrylic acid (EUDRAGIT®, Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride, or combinations thereof. Water soluble polymers also include poly(N-isopropylacrylamide)-co-poly(acrylic acid) n-butyl P(NIPAAm-co-nBu), P(NIPAAm-co-nBu) has a lower critical solution temperature (LCST) of 15° C.

Hydrophobic polymers include, but are not limited to, ethyl cellulose (EC), methylethyl cellulose (MEC), cellulose acetate (CA), cellulose acetate phthalate (CAP), cellulose acetate trimellitate (CAT), polyvinyl acetate, fatty acid, fatty acid esters, or combinations thereof.

As used herein a polymer for use in a shell or coating can include other shell materials such as wax (e.g., paraffin wax), fatty acid, and fatty acid esters, which may release reagent at higher temperature (above melting point). The other material such as wax, fatty acid, and fatty acid esters, can be used for encapsulation as native material or in combination with other polymers (e.g., cellulose acetate).

A shell herein may comprise a temperature-triggered release polymer and/or time-delayed release polymers, and optionally functional additives. In various implementations, a shell as contemplated herein comprises one or more of an anti-static agent, an anti-foaming agent, a plasticizer, one or more UCST polymers, one or more LCST polymers, hydroxypropyl methylcellulose (HPMC), or combinations thereof.

Anti-static agents include ethoxylated PEI, Kollidon® VA64, Kollicoat® IR, HEPES, MOPS and tetraalkyl ammonium chloride Efka® IO, Efka® IO 6786, Larostat 902A, Sodium lauryl sulfate, Sodium oleate, Stearic acid, Magnesium stearate, Sodium citrate tribasic dehydrate, Sodium L-Ascorbate, Span® 60, Tween® 60 polyethyleneimine, Lauric acid diethanolamide, Luviquat® FC370, Luviquat® FC550, Triglycerol monosterate, Piperidyl Sebacate, Vitamin E/Tocopherol, Trioleate Glycerohydrophobic I, Polyaniline, Coumarin 6, Tween and Makon® 17R4. (See, e.g., US20220331770 herein incorporated by reference).

Exemplary plasticizers include PEG, triethyl citrate (Citroflex 2), triacetin (glyceryl triacetate), acetyl triethyl citrate (Citroflec A2), Carbowax 400 (polyethylene glycol 400), Makon 17R4, diethyl phthalate, tributyl citrate, acetylated monoglycerides, glycerol, fatty acid esters, propylene glycol, and dibutyl phthalate. See, e.g., WO 2017/100861.

Anti-foaming agents include, but are not limited to, 13-sorbitan sesquioleate, silicone, fats, oils, waxes, aliphatic acids or esters, alcohols, sulfates, sulfonates, fatty acids, soaps, nitrogenous compounds, phosphates, polyglycols, sulfides, thio compounds, siloxanes, halogenated and inorganic compounds, ethylene oxide propylene oxide copolymer, Makon 17R4, polyether polyols such as Foamblast 882 (Emerald Foam Control), Erol DF 204K (Ouvrie PMC), DG436 (ODG Industries, Inc.), and KFO 880 (KABO Chemicals, Inc. See, e.g., U.S. Pat. No. 8,076,113

Anti-tacking agents include, but are not limited to, magnesium stearate, glycerol mono stearate, silicone oxide, titanium oxide, talc or PEG stearate.

A shell herein may comprise a pH-triggered release polymer. Exemplary pH-dependent release polymers for use in a shell include polymers that dissolve at different pH levels, e.g., from between about pH 4.5 to about pH 9, or from about pH 6 to about pH 8.5. Exemplary polymers include, but are not limited to, polymers that dissolve at or below pH 5: cellulose acetate, timellitate, cellulose acetate phthalate, PVA phthalate, HPMC phthalate; chitosan (soluble below pH 6); polymers that dissolve between pH 5 and 6 or above: Kollicoat MAE 100, Kollicoat 30D, Eudragit L100, Eudragit L100-55, Eudragit L30D-55, Eudragit L12.5, polymers that dissolve between about pH 6 and about pH 7: Eudragit L100/S100 1:1, Eudragit L/S12.5, Eudragit S100, Eudragit FS30D, Eudragit S12.5, shellac; carboxy methylcellulose (soluble above pH 7.4), or rennet casein (soluble around pH 9).

The amount of shell material includes, for example, any amount suitable to produce a desired shell result. In one implementation, the shell material is present in an amount between about 1 wt % and about 100 wt % of the shell. For example, the shell material may be present in about 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 60 wt %, 70 wt %, 80 wt %, 90 wt %, 100 wt %, of the shell, or any amount therebetween. In one implementation, the shell material is present in an amount between about 10 wt % and about 90 wt %, or between about 10 wt % and about 80 wt %, or between about 10 wt % and about 70 wt %, or between about 10 wt % and about 60 wt %, or between about 10 wt % and about 50 wt %, of the shell.

The shell, as described herein, may include one layer or a plurality of layers of varying compositions. For example, the shell may include one layer, two layers, three layers, four layers, five layers, six layers, seven layers, eight layers, nine layers, ten layers, or more than ten layers. Each of the layers may include the same or different materials from the other layers that are present in the shell.

In various implementations, the shell is between about 1 μM to 50 μM in thickness, between about 1 μM to about 30 M, between about 1 μM to about 20 μM, between about 5 μM to about 20 M, between about 3 μM to about 10 μM, or between about 4 μM to about 6 μM, e.g., about 1 μM, about 2 μM, about 3 μM, about 4 M, about 5 μM, about 6 μM, about 7 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 M, about 23 μM, about 24 μM, about 25 M, about 30 M, about 35 μM, about 40 μM, about 45 μM, or about 50 μM.

The shell as described herein, may, in one implementation, include a shell additive. The shell additive may be present in an amount between about 0.01% w/w of the shell and about 99% w/w of the shell. In one implementation, the shell additive is present in an amount between about 10%/t w/w and about 90% w/w of the shell. In one implementation, the shell additive is present in an amount between about 10% w/w and about 40% w/w. In one implementation, the shell additive is a moisture barrier material present in an amount no more than 90% w/w of the shell. In one implementation, the shell additive is present in an amount of at least 10% w/w concentration of the shell. For example, the shell additive may, in one implementation, be present in an amount between 0.1% w/w of the shell and about 15.0% w/w of the shell. For example, the shell additive may be present in an amount of about 0.01% w/w, 0.05% w/w, 0.1% w/w, 0.5% w/w, 1.0% w/w, 1.5% w/w, 2.0% w/w, 2.5% w/w, 3.0% w/w, 3.5% w/w, 4.0% w/w, 4.5% w/w, 5.0% w/w, 5.5% w/w, 6.0% w/w, 6.5% w/w, 7.0% w/w, 7.5% w/w, 8.0% w/w, 8.5% w/w, 9.0% w/w, 9.5% w/w, 10.0% w/w, 10.5% w/W, 11.0% w/w, 11.5% w/w, 12.0% w/w, 12.5% w/w, 13.0% w/w, 13.5% w/w, 14.0% w/w, 14.5% w/w, 15% w/w, or any amount therebetween. The amount of the shell additive may be adjusted to accommodate a particular reagent or combination of reagents, or to accommodate a particular microsphere composition.

Exemplary shell additives include, but are not limited to, one or more of a polymer, a copolymer, a block copolymer, a second polyvinyl alcohol (PVA), an ammonium salt, a conductivity promoter, a stearate derivative, an oleate derivative, a laurate derivative, a polyether compound, an amino acid, tocopherol acetate, piperidyl sebacate, sodium salt, a buffer, a chelating agent, imidazolium salt, polyaniline, or any combination thereof. In one implementation, the polyether compound is selected from polyethylene glycol, polypropylene glycol, a block copolymer derived from ethylene oxide (EO) and propylene oxide (PO), or any combination thereof. In one implementation, the stearate derivative or oleate derivative is selected from magnesium stearate, PEG stearate, triglycerol stearate. Span® 60, Tween® 60, glycerol trioleate, Tween@ 80, or any combination thereof. In some implementations, the amino acid is selected from one or more of leucine, isoleucine, phenylalanine, or any combination thereof. In one implementation, the polymer is neutral, cationic, or anionic. In some implementations, the sodium salt is selected from one or more of sodium chloride, sodium bisulfite, sodium citrate, or any combination thereof. In various implementations, the buffer is Trizma, Tris.HCl, or a combination thereof. In one implementation, the ammonium salt is selected from tetraalkyl ammonium chloride, tris(hydroxyethyl)alkylammonium chloride, or a combination thereof. In one implementation, the imidazolium salt is selected from 1-ethyl-3-methyl-imidazolium salt or polyquaternium or Luviquat® (copolymer of vinyl pyrrolidone and quaternized vinylimidazole) or a combination thereof. In one implementation, the shell additive comprises ammonium salt, copolymer, polyvinyl alcohol graft polyethylene glycol copolymer, polyvinyl alcohol (PVA), or any combination thereof. In various implementations, the shell additive is magnesium stearate or polyethylene glycol (PEG) stearate.

As described herein, a “core” or “core region” includes any material within the surrounding shell. In various implementations, a core comprises one or more lyophilized microspheres. In various implementations, a core comprises lyophilized beads. In various implementations, a core comprises beads made of non-lyophilized sugar or plastic, optionally wherein a reagent is coated and dried on the surface of the non-lyophilized microspheres or beads.

Lyophilization in accordance with the present disclosure includes methods in accordance with conventional techniques, which are known to those skilled in the art. Lyophilization is also referred to herein as freeze-drying. In the present disclosure, the term “lyophilize” or “lyophilizate” will be used as equivalent terms of “lyophilised”, “lyophilisate”, or “freeze-dried” e.g., with respect to a compositions, systems, or methods described herein.

Lyophilizable formulations can be reconstituted into solutions, suspensions, emulsions, or any other suitable form for administration or use. Lyophilizable formulations are typically first prepared as liquids, then frozen and lyophilized. The total liquid volume before lyophilization can be less than, equal to, or more than, the final reconstituted volume of the lyophilized formulation. Preferably, the final reconstituted volume of the lyophilized formulation is less than the total liquid volume before lyophilization. The lyophilization process is known to those of ordinary skill in the art, and typically includes sublimation of water from a frozen formulation under controlled conditions.

Lyophilized formulations can be stored at a wide range of temperatures. Lyophilized formulations may be stored below 25° C. for example, refrigerated at 2-8° C. or at room temperature (e.g., approximately 25° C.). Lyophilized formulations are stored below about 25° C., more preferably, at about 4-20° C.; below about 4° C.; below about −20° C.; about −40° C.; about −70° C., or about −80° C. Stability of the lyophilized formulation may be determined in a number of ways known in the art, for example, by visual appearance of the microsphere and/or cake and/or by moisture content. The compositions of the present disclosure can also withstand temperature excursions that might occur during shipping, for example, up to 70° C., for brief periods of time.

Lyophilized formulations are typically rehydrated (interchangeably referred to herein as “reconstituted”) for use by addition of an aqueous solution to dissolve the lyophilized formulation. A wide variety of aqueous solutions can be used to reconstitute a lyophilized formulation including water, saline, or another electrolyte or non-electrolyte diluent. Lyophilized formulations may be rehydrated with a solution comprising water (e.g., USP WFI, or water for injection) or bacteriostatic water (e.g., USP WFI with 0.9% benzyl alcohol). However, solutions comprising additives, buffers, excipients, and/or carriers can also be used and are described herein.

Freeze-dried or lyophilized formulations are typically prepared from liquids, that is, from solutions, suspensions, emulsions, and the like. Thus, the liquid that is to undergo freeze-drying or lyophilization preferably may comprise all components desired in a final reconstituted liquid formulation. As a result, when rehydrated or reconstituted, the freeze-dried or lyophilized formulation will render a desired liquid formulation upon reconstitution. A core additive and/or shell additive, when present, may be integrated into the reagent upon rehydration of the compositions described herein.

In one implementation, the core includes, but is not limited to, one or more reagents, for example, one or more enzyme, salt, surfactant, buffering agent, enzyme inhibitor, primer, nucleotide, organic osmolite, magnetic bead, molecular probe, crowding agent, small molecule, labelled-nucleotide, a fluorophore, or any combination thereof.

As used herein, the term “reagent” describes a single agent or a mixture of two or more agents useful for reacting with, interacting with, diluting, or adding to a sample, and may include agents used in nucleic acid reactions, including, for example buffers, chemicals, enzymes, polymerase, primers including those having a size of less than 60 base pairs, template nucleic acids, nucleotides, labels, dyes, or nucleases. A reagent as described herein may, in certain implementations, include enzymes such as polymerases, ligases, recombinases, or transposases; binding partners such as antibodies, epitopes, streptavidin, avidin, biotin, lectins or carbohydrates; or other biochemically active molecules. Other exemplary reagents include reagents for a biochemical protocol, such as a nucleic acid amplification protocol, an affinity-based assay protocol, an enzymatic assay protocol, a sequencing protocol, and/or a protocol for analyses of biological fluids. According to some implementations disclosed herein, a reagent may include one or more beads, in particular magnetic beads, depending on specific workflows and/or downstream applications.

In one implementation, a reagent in accordance with the present disclosure is a polymerase. Polymerase in accordance with the present disclosure may include any polymerase that can tolerate incorporation of a phosphate-labeled nucleotide. Examples of polymerases that may be useful in accordance with the present disclosure include but are not limited to Q5 polymerase, tTaq608, phi29 polymerase, a klenow fragment, DNA polymerase I, DNA polymerase III, GA-1, PZA, phi15, Nf, G1, PZE, PRD1, B103, GA-1, 90N polymerase, Bst, Bsu, T4, T5, T7, Taq, Vent, RT, pol beta, and pol gamma. Polymerases engineered to have specific properties may be used.

A primer as disclosed herein includes a nucleic acid molecule that can hybridize to a target sequence of interest. In various implementations, a primer may function as a substrate onto which nucleotides can be polymerized by a polymerase. However, in some examples, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some implementations, the primer is a single-stranded oligonucleotide or polynucleotide.

Non-limiting examples of nucleic acid molecules that may be encapsulated within a microsphere include those described above, for example, DNA, such as genomic or cDNA; RNA, such as mRNA, sRNA or rRNA; or a hybrid of DNA and RNA. The nucleic acid molecule may further comprise a labelled-nucleotide.

The term “salt” may include salts prepared from toxic or non-toxic acids or bases including inorganic acids and bases and organic acids and bases. Salts may be prepared from, for example, pharmaceutically acceptable non-toxic acids including inorganic and organic acids. Salts useful as reagents herein include NaCl, KCl, MgCl2, and/or MgSO4.

Any surfactant known to one skilled in the art may be used as a reagent in the microsphere. The surfactant may be non-ionic or ionic (specifically cationic or anionic) or may be zwitterionic. Examples of suitable surfactants include but are not limited to polyacrylate surfactants, silicone surfactants, and/or other commercially available surfactants or detergents. Examples of cationic surfactants are cetyldimethylammonium acetamide, octadecyl-dimethylammonium acetamide, tetradecyl-dimethylammonium acetamide, dodecyl-dimethylammonium acetamide, cetyltrimethylammonium, octadcecyl-trimethylammonium, tetradecyl-trimethylammonium, dodecyl-trimethylammonium, dimethyldioctadecylammonium, dioctadecyldimethylammonium, and mixtures thereof. Suitable sources of these cations of the cationic surfactant include, but are not limited to, alkyltrimethylammonium salts: such as cetyl trimethylammonium bromide (CTAB) or cetyl trimethylammonium chloride (CTAC); cetylpyridinium chloride (CPC); dimethyldioctadecylammonium chloride; dioctadecyldimethylammonium bromide (DODAB); cetyldimethylammonium acetamide bromide; or other cationic surfactant alike, including lipids. Alternatively, the surfactant may be benzyl hexadecyl dimethyl ammonium chloride (BHDC). The core may include an anionic surfactant which contains an anionic functional group at one end, such as a sulfate, sulfonate, phosphate, and carboxylate functional group. One example of an anionic surfactant is sodium dodecyl sulfate. The core may comprise a neutral surfactant, for example, a polyethylene glycol lauryl ether.

A microsphere may further, or in the alternative, include an enzyme inhibitor, a molecular probe, a crowding agent, organic osmolite, cyclodextrin, adenosine triphosphate (ATP), ethylenediaminetetraacetic acid (EDTA), creatine kinase, creatine phosphate, palladium, lipoic acid, hexaethylene glycol, trihydroxypropanephosphine, sodium ascorbate, or any combination thereof. An enzyme inhibitor as described herein includes any molecule that binds to an enzyme and decreases its activity, e.g., proteinase K or sodium dodecyl sulfate. A molecular probe as described herein includes, for example, digoxigenin, 8-Anilinonaphthalene-1-sulfonic acid (“ANS”), porphyrin, BODIPY, cyanine, or any combination thereof. A crowding agent as described herein includes any crowding agent known to those skilled in the art. Examples include, but are not limited to, polyethylene glycol, ficoll, dextran, and serum albumin.

In various implementations, the one or more tagmentation reagents comprises a bead linked transposomes (BLT), primers, probes, transposase, ligase, buffers, divalent cations, Tris buffer, cobalt buffer, and/or a lyophilization reagent. In various implementations, the one or more tagmentation reagents comprises a bead linked transposomes (BLT), transposase, primers, buffers, Tris buffer, cobalt buffer, and/or a lyophilization reagent. In various implementations, the tagmentation reagents comprise bead linked transposomes, primers, cobalt chloride and trehalose. In various implementations, the tagmentation reagents comprise bead linked transposomes, magnesium sulfate, Tris buffer and trehalose.

In various implementations, the one or more tagmentation poisoning reagents includes a chelator, salts, primers, polymerase (e.g., Q5, tTaq), co-enzyme (e.g., NAD₊), deoxynucleotide triphosphates (dNTPs), Tris buffer, potassium buffer, ammonium salts, and/or a lyophilization reagent. In various implementations, the tagmentation poisoning reagents comprise potassium chloride, ammonium sulfate, cyclen tetrahydrochloride, alpha cyclodextrin, hydroxypropyl-beta-cyclodextrin (HPBCD), dNTPs and/or trehalose. In various implementations, the tagmentation poisoning reagents comprise Tris buffer, potassium chloride, ammonium sulfate, cyclen tetrahydrochloride, alpha cyclodextrin, dNTPs and/or trehalose. In various implementations, the tagmentation poisoning reagents comprise Tris buffer, potassium chloride, sodium dodecyl sulfate, dNTPs and trehalose. In various implementations, the tagmentation poisoning reagents comprise potassium chloride, ammonium sulfate, SDS, dNTPs and trehalose.

In various implementations, the extension-ligation and PCR reagents comprise one or more of magnesium buffer, polymerase (e.g., Q5, tTaq), co-enzyme (e.g., NAD₊), deoxynucleotide triphosphates (dNTPs), ligase, primers, probes, divalent cations, Tris buffer, ammonium salts, betaine, a surfactant, or lyophilization reagent. In various implementations, the extension-ligation and PCR reagents comprise one or more of magnesium sulfate, glycerol free hot start Q5, tTaq 608 polymerase, ampligase, NAD₊, betaine, Tween-20 and/or trehalose. In various implementations, the extension-ligation and PCR reagents comprise alpha-cyclodextrin, glycerol free hot start Q5, tTaq 608 polymerase, ampligase, NAD₊, betaine, Tween-20 and/or trehalose. In various implementations, the extension-ligation and PCR reagents comprise glycerol free hot start Q5, tTaq 608 polymerase, ampligase, NAD₊, betaine, alpha cyclodextrin, Tween-20 and trehalose.

Those skilled in the art of sequencing technologies will appreciate there are additional reagents that may be useful in the compositions, systems, and methods of the present disclosure that are not explicitly described herein.

A core as described herein may, in one implementation, further include one or more additional agents. The one or more additional agent in the core improves the ability to control the release of one or more lyophilized microspheres. In one implementation, the additional agent is selected from one or more sugars, amino acids, polymers, mesoporous silica, quaternary amines, lyophilization reagents, or any combination thereof.

Lyophilization reagents, (which may be also called excipients or cryoprotective agents, lyoprotectants or cryoprotectants) contribute to the preservation of the structures of proteins, liposome bilayers and other substances during freezing in general. Lyoprotectants stabilize these substances during drying, especially freeze-drying. In freeze-drying lyoprotectant may be also considered as a cryoprotectant, so as used herein the term “cryo-protectant” may also include lyoprotectants or pyophilization reagents. Examples of lyoophilizatino reagents include polyhydroxy compounds such as sugars (mono-, di-, and polysaccharides), trehalose and sucrose as natural lyoprotectants and polyalcohols, such as glycerol, mannitol, sorbitol, and their derivatives.

In one implementation the cryoprotective and/or lyoprotective agent(s) is selected from the group consisting of trehalose, sucrose, mannitol, maltose, maltodextrin, dextran, cyclodextrin, inulin, raffinose, sorbitol, inositol, glucose, and mannose. In one implementation the cryoprotectant is trehalose. Trehalose, also known as α,α-trehalose; a-D-glucopyranosyl-(1→1)-a-D-gluco-pyranoside, mycose or tremalose, is a natural alpha-linked disaccharide formed by an a, a-1,1-glucoside bond between two a-glucose units. Trehalose may be present as anhydrous or as dihydrate.

In one implementation, when the additional agent comprises a lyophilization reagent, it may be a sugar, wherein the sugar is selected from trehalose, mannitol, cyclodextrin, dextran, sucrose, or any combination thereof. In another implementation, when the additional agent comprises an amino acid, the amino acid has a hydrophobic side chain. In another implementation, when the additional agent comprises a polymer, the polymer is selected from poly vinylpyrrolidone, polyvinyl alcohol, or a combination thereof. In some implementations, the additional agent may be, for example, one or more co-polymers, ionic liquids, or any combination thereof. The additional agent may be added in any amount suitable to produce a desired effect, for example, between about 0.1 wt % and about 50 wt % of the core. In one implementation, the concentration of the additional agent in the core is about 0.1 wt %, 0.5 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 20 wt %, 25 wt %, 30 wt %, 35 wt %, 40 wt %, 45 wt %, or any amount therebetween.

The composition (i.e., comprising lyophilized microspheres or beads, whether encapsulated or not encapsulated) may be any appropriate size or volume that is appropriate to encompass one or more reagents and suitable for use in library preparation for sequencing. In one implementation, the composition has a volume of reagent in a core region of between about 0.1 μL and about 50 μL. For example, the composition (i.e., comprising lyophilized microspheres, encapsulated or not encapsulated) may have an active reagent volume of about 0.1 μL, 0.5 μL, 1 μL, 2 μL, 3 μL, 4 μL, 5 μL, 6 μL, 7 μL, 8 μL, 9 μL, 10 μL, 15 μL, 20 μL, 25 μL, 30 μL, 35 μL, 40 μL, 45 μL, 50 μL, or any volume between about 0.1 μL and about 50 μL. In one implementation, the active reagent volume is between about 10 μL and about 40 μL. The composition (i.e., lyophilized microsphere, encapsulated or not encapsulated) may have a diameter of, for example, about 2 μm to about 120 μm, such as, for example, 2, 3, 4, 5, 6, 7, 8, 9, 10, 15, 20, 25, 30, 35, 40, 45, 50, 60, 70, 80, 90, 100, 110, or 120 μm in diameter, or a diameter within a range defined by any two of the aforementioned values.

The composition described herein may include an additional reagent in the shell of the microsphere. In one implementation, the encapsulated microsphere includes a reagent or additive in the microsphere shell. The reagent in the shell may include, for example, any of the foregoing reagents or additives. In one implementation, the shell contains no nucleic acid molecules, for example, the shell contains no DNA. In one implementation, the shell contains more than one reagent and, or in the alternative, more than one additive.

The compositions described herein (i.e., comprising lyophilized microspheres, beads or cakes, whether encapsulated or not encapsulated) may be used for multiple sequential co-assays comprising lysis, DNA analysis, RNA analysis, protein analysis, tagmentation, nucleic acid amplification, nucleic acid sequencing, DNA library preparation, SBS technology, assay for transposase accessible chromatic using sequencing (ATAC-seq), contiguity-preserving transposition (CPT-seq), or any combination thereof performed sequentially. In one implementation, the composition is used for performing multiple co-assay reactions. The compositions, systems, and methods described herein may improve sequencing quality, enable one-pot library prep, and simplify manufacturing. As used herein, the term “one-pot reaction” may also be referred to as “transfer-free reaction.”

The compositions, systems, and methods described herein may be prepared for various stages of sequencing including, but not limited to, sample extraction, library preparation, enrichment, and sequencing. In sample extraction compositions, the core may include enzymes, salt, surfactants, buffering agents, and any combination thereof. The sample extraction may occur at a pH of about 7.5 with a reaction volume of between about 1 mL and about 5 mL. In library preparation compositions, the lyophilized microspheres and/or core may include enzyme inhibitors, salts, primers, enzymes, nucleotides, organic osmolites, magnetic beads, and any combination thereof. Library preparation may occur at a pH of about 8 with a reaction volume of about 0.05 mL. In enrichment compositions, the lyophilized microspheres and/or core may include nucleotides, molecular probes, enzymes, magnetic beads, crowding agents, and any combination thereof. Enrichment may occur at a pH of about 8.5 with a reaction volume of between about 0.1 mL and about 0.2 mL. In clustering compositions, the lyophilized microspheres and/or core may include salts, enzymes, one or more nucleotides, small molecules, surfactants, primers, and any combination thereof. Clustering may occur at a pH of about 8.6 with a reaction volume of between about 1 mL and about 5 mL. In sequencing compositions, the lyophilized microspheres and/or core may include labelled-nucleotides, a fluorophore, surfactants, salts, enzymes, small molecules, and any combination thereof. Sequencing may occur at a pH of between about 7 and about 10 with a reaction volume of about 30 mL to about 100 mL.

In various implementations, the lyophilized microspheres and/or shell may rehydrate under a pH between 1 and 14. In one implementation, the shell may include one or more shell layers and each layer may rehydrate under the same or different conditions. For example, the shell may include a plurality of layers that rehydrate under different conditions. In one implementation, the shell may include two or more layers (e.g., three or more layers) that release at different pH levels, for example, one layer may release at a pH of 5, one layer may release at a pH of 5.5, one layer may release at a pH of 6, one layer may release at a pH of 6.5, one layer may release at a pH of 7, one layer may release at a pH of 7.5, and/or one layer may release at a pH of 8.

In various implementations, a biological sample contacts the composition. A biological sample, may include, for example, whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. A biological sample can include nucleic acids, such as DNA, genomic DNA, RNA, mRNA or analogs thereof, nucleotides such as deoxyribonucleotides, ribonucleotides or analogs thereof such as analogs having terminator moieties such as those described in Bentley et al., “Accurate Whole Human Genome Sequencing Using Reversible Terminator Chemistry,” Nature 456:53-59 (2008); WO/2013/131962; U.S. Pat. No. 7,057,026; WO/2008/042067; WO/2013/117595; U.S. Pat. Nos. 7,329,492; 7,211,414; 7,315,019; 7,405,281; and U.S. Patent Pub. No. 20080108082, all of which are hereby incorporated by reference in their entirety.

Any suitable method can be used to form microspheres. Standard microsphere manufacturing techniques will be known to those skilled in the art, and include, preparing frozen pellets and placing those pellets in a dryer as described herein. A variety of microspheres are contemplated in accordance with the compositions, systems, and methods of the present disclosure and include, for example, time sustained release, immediate pulse, timed pulsative release, organic acid Diffucaps® Bead, and alkaline buffer Diffucaps® Bead microspheres. There are also a variety of types of encapsulation encompassed by the compositions, systems, and methods described herein, including, but not limited to, bulk-, micro-, nano-, single molecule, and ionic encapsulation.

Modifications to standard microsphere production may be used to manufacture the compositions described herein. For example, one or more additional feed buffer tanks and one or more suitable nozzles and/or nozzle plates may be added to standard microsphere production equipment. Other modifications may be made, in particular, to a solidification system, to produce various types of shells and include compounds such as hydrocolloids, alginate, and pectin among others as described herein.

A rehydration (or reconstitution) solution, e.g., a sample with DNA, as used herein may include water, deionized water, saline solutions, acidic solutions, basic solutions, detergent solutions and/or buffers. In a preferred implementation, the rehydration solution is water or buffer. Additional additives as described herein may be provided in the rehydration solution to further improve control of release of microspheres.

In various implementations, a pH in the rehydration solution is between about 6.0 and about 10.0, or between about 7.0 and about 8.0. A pH of the rehydration solution may be, for example, about 6.0, about 6.5, about 7.0, about 7.5, about 8.0, about 8.5, about 9.0, about 9.5, about 10.0, or any amount therebetween. Rehydration time will vary depending on composition content and reaction conditions (e.g., reagents, temperature, pH). In various implementations, rehydration time may be between 0.1 seconds and 10 hours. For example, rehydration time may be about 0.1 seconds, 1 second, 10 seconds, 30 seconds, 45 seconds, 60 seconds, 5 minutes, 10 minutes, 12 minutes, 15 minutes, 20 minutes, 30 minutes, 40 minutes, 50 minutes, 60 minutes, 70 minutes, 80 minutes, 90 minutes, 2 hours, 5 hours, 8 hours, 10 hours, or any amount of time therebetween.

In some implementations, the first trigger release condition is effective to release a lyophilized microsphere (e.g., a second lyophilized microsphere as described in the composition or system). The lyophilized microsphere is in accordance with the lyophilized microspheres and compositions described herein.

In some implementations, the second trigger release condition is effective to release a lyophilized microsphere (e.g., a third lyophilized microsphere as described in the composition or system), wherein contents of the third lyophilized microsphere are different from content of the second lyophilized microsphere. The third lyophilized microsphere is in accordance with the lyophilized microspheres and compositions described herein.

In some implementations, the first trigger release condition is effective to release a lyophilized microsphere (e.g., a second lyophilized microsphere as described in the composition or system) and is preceding by a second release trigger mechanism that initiates the first trigger release condition. The lyophilized microsphere is in accordance with the lyophilized microspheres and compositions described herein.

In some implementations, the third trigger release condition is effective to release a lyophilized microsphere (e.g., a third lyophilized microsphere as described in the composition or system), wherein contents of the third lyophilized microsphere are different from content of the second lyophilized microsphere. The third lyophilized microsphere is in accordance with the lyophilized microspheres and compositions described herein.

One way to enable sequential release of lyophilized reagents is through temperature triggered release, for example, by dipping particles comprising a first or second shell and filled with microspheres in paraffin wax or another shell agent as described herein. Similarly, such an approach enables a time-triggered release by addition of additives to a rehydration solution, for example, amino acids. Other reaction characteristics may be modified in addition to or instead of time and, or in the alternative, temperature. For example, pH and humidity may be modified to further control release of one or more encapsulated microspheres and the reagent(s) contained therein.

In one example, two compositions having a shell layer and inner core, respectively (e.g., particles) may be in a single container, and those particles may dissolve with different triggers. In another example, two or more particles may be in the container and a second particle is dissolved upon release of a first particle when in contact with a liquid, followed by dissolving of a third particle upon release of the second capsule, when in contact with a liquid, which is repeated for as many particles as are present in a particular container. These implementations may likewise be triggered by other modifications such as temperature change (heat or cooling), or pH.

Provided herein are systems comprising the compositions as described herein. The system includes one or more compositions as described herein, and one or more containers, wherein the one or more composition is placed in one or more containers under conditions effective to form a sequential system for preparing a DNA library. In various implementations, the system comprises one more containers to hold the composition, wherein the one or more containers include a PCR tube, vial, microtube, flow cell, multiwell plate, glass tube, transwell membrane/mesh insert, cartridge or microfluidic chip.

The system may further include a temperature controller or sensor. The temperature controller may be used to change or adjust temperature of the system to further control release of various components of the compositions described herein. For example, the temperature controller may be used to speed up or slow down the release from the first or second shell. Similarly, the temperature controller may be used to speed up or slow down the release of the interior core to facilitate or control the release of one or more reagents. In one implementation, the system comprises a temperature controller on the container in the system. For example, the temperature controller may include a resistive heater proximate to a wall of the container, e.g., a cartridge, tube, chip or well, to provide heat thereto. The temperature controller may also include a temperature sensor. The temperature controller may also include circuitry to activate and deactivate the heater to maintain the well at a specified temperature.

Methods of Generating a DNA library

Also contemplated herein is a method of generating a DNA library preparation. The method includes providing compositions as described herein and mixing the compositions with a solution under a first trigger condition effective to control release of reagent from a lyophilized substance, and or control release of one or more lyophilized microspheres or lyophilized beads from the core of a composition.

Methods contemplated herein are carried out in accordance with the previously described implementations, in particular with regard to the characteristics of the one or more lyophilized microspheres or lyophilized beads and their shell, core, and/or encapsulation.

In various implementations, provided are methods of preparing a DNA library comprising: adding a sample comprising DNA to a container comprising a composition or system as described herein, wherein addition of the sample initiates dissolution of the first lyophilized substance to release one or more tagmentation reagents into the container to initiate a tagmentation reaction;
allowing the tagmentation reagents to continue under conditions and for a sufficient period to complete tagmentation of the DNA in the sample;
initiating the first trigger release condition, wherein the first trigger release condition of the first shell releases one or more tagmentation poisoning reagents of the first core into the container to initiate a tagmentation poisoning reaction; and
allowing the tagmentation poisoning reaction to continue under conditions and for a sufficient period to initiate the second trigger release condition of the second shell, wherein one or more extension ligation and PCR reagents are released from the second core, wherein the extension-ligation and PCR reagents carry out gap fill and PCR amplification.

In various implementations, the method further comprises isolating the DNA and sequencing the DNA library.

In various implementations, the tagmentation reaction is carried out between 37° C. to 42° C., for example, at 37° C., 38° C., 39° C., 40° C., 41° C., or 42° C.

In various implementations, the tagmentation reaction is carried out for up to 15 minutes, for example, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, or 15 minutes.

In various implementations, the tagmentation reaction is carried out between 37° C. to 41° C. for 5 minutes.

In various implementations, the first release condition is a temperature release condition, pH release condition, or time release condition. In various implementations, the first release condition is a temperature release condition.

In various implementations, the temperature for the first temperature release condition is equal to or above about 45° C., e.g., 45° C., 46° C., 47° C., 48° C.49° C., 50° C., 51° C., 52° C., 53° C., 54° C., 55° C., 56° C., 57° C., 58° C., 59° C., 60° C., 61° C., 62° C., 63° C., 64° C., or 65° C. In various implementations, the first release condition is met for up to about 10 minutes, for example, about 1 minute, about 2 minutes, about 3 minutes, about 4 minutes, about 5 minutes, about 6 minutes, about 7 minutes, about 8 minutes, about 9 minutes or about 10 minutes. In various implementations, the condition for the first trigger release condition is about 45° C., for about 5 minutes.

In various implementations, the tagmentation poisoning reaction is carried out between about 45° C. to about 65° C., for example, about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C. or about 65° C. In various implementations, the tagmentation poisoning reaction is carried out between about 60° C. to about 65° C., for example, at about 60° C., about 61° C., about 62° C., about 63° C., about 64° C. or about 65° C.

In various implementations, the tagmentation poisoning reaction is carried out for up to about 10 minutes, for example, about 1 minute, about 2 minutes, about 3 minutes, about 4 minutes, about 5 minutes, about 6 minutes, about 7 minutes, about 8 minutes, about 9 minutes or about 10 minutes.

In various implementations, the tagmentation poisoning reaction is carried out at between about 60° C. to 65° C. for about 1 to 10 minutes.

In various implementations, the second release condition is a temperature release condition, pH release condition, or time release condition. In various implementations, the second release condition is a time release condition.

In various implementations, the time release condition is between about 1 minute and about 15 minutes, or between about 5 minutes and about 15 minutes, e.g., about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In various implementations, the time release condition is between about 1 minute and about 15 minutes, or between about 5 minutes and about 15 minutes at pH and/or at between about 45° C. and about 60° C. In various implementations, the time release condition is about 1 minute, about 2 minutes, about 3 minutes, about 4 minutes, about 5 minutes or about 6 minutes at between about 45° C. and about 60° C.

In various implementations, the time release condition is met when the tagmentation poisoning reaction is cooled to about 50° C. for about 1 minute to about 10 minutes.

In various implementations, the time release condition is from about 1 minute to about 3 minutes at about 50° C.

Sequencing of a DNA library can be carried out using any suitable sequencing technique, and methods for determining the sequence of amplified adapter-target-adapter molecules are known in the art and are described in, for instance, U.S. Pat. No. 8,053,192, WO2016/130704, U.S. Pat. Nos. 8,895,249, and 9,309,502.

The compositions, systems and methods described herein for DNA library preparation may also be used within a workflow for other methods of DNA library preparation, such as spatial indexing, sequencing by synthesis (SBS) and other next-generation sequencing (NGS) technologies. Compositions comprising lyophilized substances as described herein are adapted to comprise reagents, e.g., tagmentation reagents, extension-ligation reagents, for use in a spatial indexing workflow, and may be added to an array or substrate as appropriate for carrying out the desired reactions.

Exemplary SBS systems and methods are described in U.S. Pat. Publ. No. 2007/0166705, 2006/0188901, 2006/0240439, 2006/0281109, 2012/0270305, and 2013/0260372, U.S. Pat. No. 7,057,026, WO 05/065814, U.S. Pat. Publ. No. 2005/0100900, WO 06/064199, and WO 07/010,251, U.S. Pat. Publ. No. 2013/0079232.

For the ligation reaction, multiple ligases may prove useful in the method. In various implementations, the polymerase is T4 DNA ligase, T4 RNA Ligase 2 (T4Rnl2), SplintR DNA ligase, E. coli DNA ligase, Ampligase or R2D LIGASE. In various implementations, the ligation reaction is carried out between 16° C. to 50° C., between 25° C. to 45° C., or between 30° C. to 42° C. In various implementations, the ligation reaction is carried out at 37° C.

In various implementations, the isolated PCR templates are placed in a tube for DNA library preparation.

In some implementations, the DNA in the sample undergoes a PCR clean-up reaction. In various implementations, PCR-clean up includes use of double solid phase reversible immobilization (dSPRI) for PCR clean-up to remove small and large DNA fragments, as well as removed of unused primers and bases.

### Kits

Kits and articles of manufacture are also contemplated herein. Such kits can comprise a carrier, package, or container that is compartmentalized to receive one or more containers such as vials, tubes, and the like, each of the container(s) comprising one of the separate elements to be used in a method described herein. Suitable containers include, for example, PCR tubes, vials, microtubes, flow cells, multiwell plates, e.g., a 96- or 384-well plate, glass tubes, transwell membrane/mesh inserts, cartridges or microfluidic chips. The containers can be formed from a variety of materials such as glass or plastic. For example, the container(s) can comprise one or more spatially addressable probes disclosed herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use with the spatially addressable probes described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific applications. The label can also indicate directions for use of the contents, such as in the methods described herein.

In the present disclosure, reference is made to the accompanying drawings that form a part hereof, and in which is shown by way of illustration specific implementation which may be practiced. These examples are described in detail to enable those skilled in the art to practice the disclosure, and it is to be understood that other implementations may be utilized and that structural, logical and electrical changes may be made without departing from the scope of the present disclosure. The following description of examples is, therefore, not to be taken to be limiting.

The present disclosure may be further illustrated by reference to the following examples.

### EXAMPLES

### Example 1

In an experiment to determine if lyophilized microspheres may help reduce the number of steps in DNA library preparation and improve overall workflow processes, reagents in a DNA library preparation protocol are lyophilized and their activity compared to non-lyophilized reagents in the same workflow.

A mixture of tagmentation reagents comprising bead linked transposomes and cobalt chloride is mixed with trehalose and lyophilized to form lyophilized microspheres (Table 1). In a second formulation, bead linked transposomes, Tris buffer and magnesium sulfate are mixed with trehalose and lyophilized.

TABLE 1
Tagmentation Microsphere Formulations

| Tagment buffer (2X) | Conc | units |
|---|---|---|
| Bead Linked Transposome | 20 | uL |
| Cobalt Chloride | 5-10 | mM |
| Trehalose | 550 | mM |
| Total solute content (%) | | 19.96 |
| Bead Linked Transposome | 20 | uL |
| Tris HCl, pH 7.5 | 20 | mM |
| Magnesium sulfate | 4 | mM |
| Trehalose | 560 | mM |
| Total solute content (%) | | 20.53 |

A second group of reagents is admixed in a particle having a shell and an inner core to form a tagmentation poisoning buffer. In one implementation, the core comprises potassium chloride, ammonium sulfate, cyclen tetrahydrochloride, dNTPs and trehalose in approximately 25 μL. In another formulation, the core comprises potassium chloride, ammonium sulfate, SDS, dNTPs and trehalose.

TABLE 2
Tagmentation Poison Microsphere Formulations

| Tn5 poison (5X) | Conc | units |
|---|---|---|
| Potasium chloride | 375 | mM |
| Tris, pH 8.8 | 475 | mM |
| Ammonium sulphate | 50 | mM |
| Cyclen tetrahydrochloride | 37.5 | mM |
| dNTPs | 1 | mM |
| Trehalose | 400 | mM |
| Total solute content (%) | | 41.17 |
| Potasium chloride | 375 | mM |
| Ammonium sulphate | 50 | mM |
| SDS | 0.5 | % |
| dNTPs | 1 | mM |
| Trehalose | 750 | mM |
| Total solute content (%) | | 29.68 |

The shell comprises temperature-triggered release polymers, like UCST polymers, i.e. poly(acrylonitrile-co-acrylamide) and poly(N-acryloylglycinamide-co-styrene) or LCST polymers, i.e. poly(N-isopropylacrylamide). As an alternative, the shell comprises wax or a mixture of time release polymer, i.e., HPMC, cellulose acetate, and wax, i.e. paraffin, myristic acid. In one implementation, the shell comprises polymeric material Efka 6783, Makon 17R4, magnesium stearate and an upper critical soluble temperature (UCST) polymer 1 (e.g., poly(acrylonitrile-co-Acrylamide) or poly(N-acryloylglycinamide-co-Styrene). The reagents for the core are lyophilized resulting in lyophilized microspheres which are then encapsulated within the shell. The shell may also comprise PEG stearate.

A third group of reagents used in extension-ligation and PCR reactions for DNA library preparation is admixed in a shell and core format to form particles comprising lyophilized microspheres. The extension-ligation and PCR reagents magnesium sulfate, glycerol free hot start Q5, tTaq 608 polymerase, ampligase, NAD₊, betaine, Tween-20, HPBCD and trehalose are lyophilized. In another formulation, extension-ligation and PCR reagents Tris buffer, glycerol free hot start Q5, tTaq 608 polymerase, ampligase, NAD₊, betaine, Tween-20, αCD and trehalose are lyophilized. The lyophilized microspheres are encapsulated in double shells comprising a time release polymer, i.e. HPMC, cellulose acetate as the inner shell and a temperature release polymer as the outer shell, i.e., wax, UCST or LCST polymers. Functional additives, such as an anti-tacking agent, anti-static agent, anti-foaming agent and plasticizer can be incorporated into the polymer mixture comprising Efka 6783, Makon 17R4, magnesium stearate and UCST polymer 2 (e.g., poly(acrylonitrile-co-Acrylamide) or poly(N-acryloylglycinamide-co-Styrene) and hypromellose (Table 3).

TABLE 3
Extension-Ligation and PCR Mix Microsphere Formulations

| Extension-Ligation and PCR (2X) | Conc | units |
|---|---|---|
| Magnesium sulphate | 4 | mM |
| NAD+ | 1 | mM |
| Betaine | 2000 | mM |
| Tween 20 | 0.2 | % |
| glycerol free Hot start Q5 Pol | 4 | uL |
| tTaq 608 Pol | 1 | uL |
| Ampligase | 0.2 | uL |
| HPBCD | 100 | mM |
| Trehalose | 100 | mM |
| Total solute content (%) | | 42.52 |
| Tris, pH 8.8 | 190 | mM |
| NAD+ | 1 | mM |
| Betaine | 500 | mM |
| Tween 20 | 0.2 | % |
| glycerol free Hot start Q5 Pol | 4 | uL |
| tTaq 608 Pol | 1 | uL |
| Ampligase | 0.2 | uL |
| alpha-cyclodextrin | 4.5 | % |
| Trehalose | 336 | mM |
| Total solute content (%) | | 24.43 |

The lyophilized microspheres containing tagmentation reagents, and particles comprising the tagmentation poisoning reagents or the extension-ligation and PCR mix, respectively, are placed in a single PCR tube to carry out the initial library preparation experiments. In certain implementations, stabilizers used in the extraction include alkyl polyglucosides, and/or PEG 6K.

A sample comprising genomic DNA (50 μL, approximately 150 ng DNA) is inserted into the tube and the aqueous solution reconstitutes the lyophilized microspheres comprising the tagmentation reagents. The tagmentation reaction is allowed to run 5 minutes at 37° C. to complete fragmenting of the genomic DNA and addition of DNA adaptors (e.g., A14 and B15) (see, e.g., US20180245069, incorporated by reference) onto the DNA fragments. To stop the tagmentation reaction and remove the Tn5 transposon from the fragmented DNA, the temperature is raised to 65° C. for 1 minute. The increase in temperature triggers dissolution of the shell on the poisoning microsphere particles and release of the tagmentation poisoning reagents from the core into the PCR tube. The removal of Tn5 is completed and the DNA samples are ready for extension-ligation and PCR. The temperature of the mixture is lowered to 50° C. which triggers the dissolution of the shell for the extension-ligation and PCR mix microsphere particles and the core releases its contents into the PCR tube to carry out gap fill and ligation.

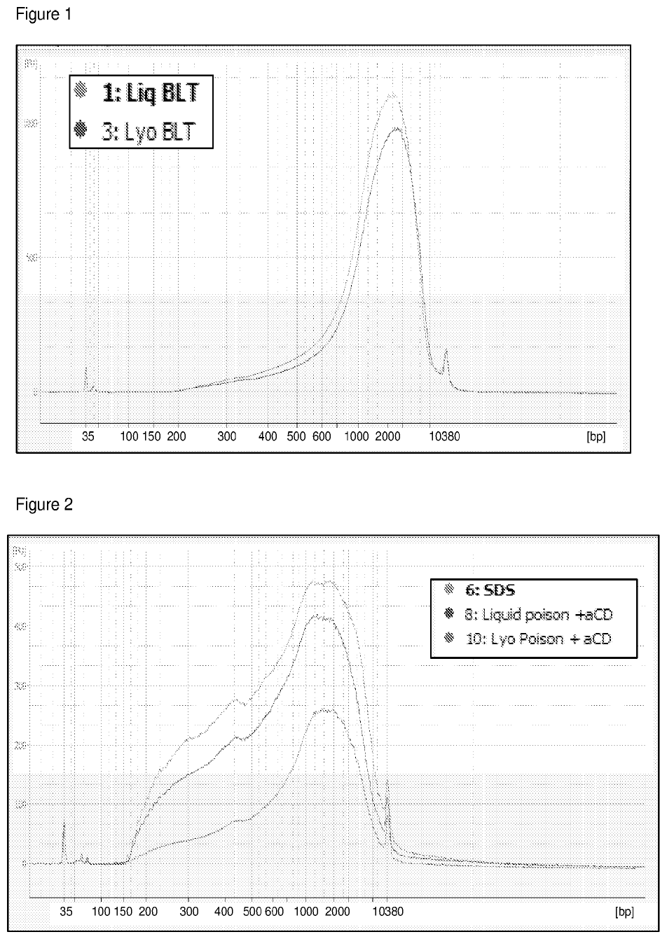

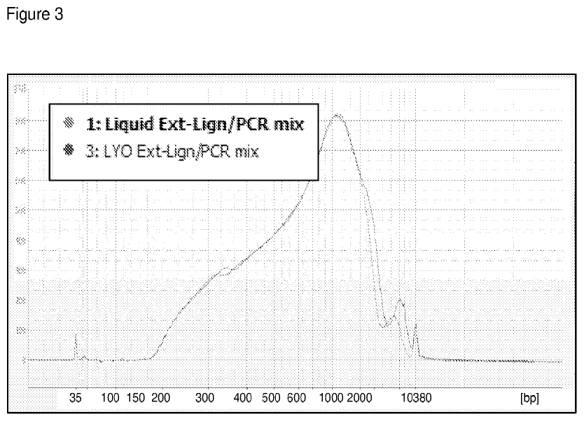

A comparison of the tagmentation reaction using either liquid or lyophilized reagents shows that there is essentially no difference in the outcome of the reaction when lyophilized reagents are used for tagmentation (FIG. 1). Comparison of the tagmentation poisoning reagents when lyophilized or in liquid form shows that there may be a slightly lower DNA yield when using lyophilized buffers (FIG. 2). For the PCR mix reactions, the lyophilized and liquid forms of the reagents yield similar results (FIG. 3).

### Example 2

When multiple microsphere release conditions are utilized in a single pot assay, this may benefit from temperature and pH changes to accommodate the different release conditions. These changes risk that by cooling the reaction or altering the pH, material may become insoluble and precipitate into the reaction mixture and interfere with the process.

In another workflow design, tagmentation is carried out at 55° C. and poisoning is carried out at 60° C. in order to reach the first shell release condition in the reaction mixture, wherein the shell is a UCST polymer. To proceed through the extension and ligation steps, the reaction is lowered to 45° C. and the second shell release condition, e.g., time, pH or temperature release, is met, wherein the second shell is a LCST polymer. If PCR is subsequently carried out under PCR cycling conditions, e.g., heating to 98° C. and cooling to 55° C., there can be precipitation of the polymeric material out of solution.

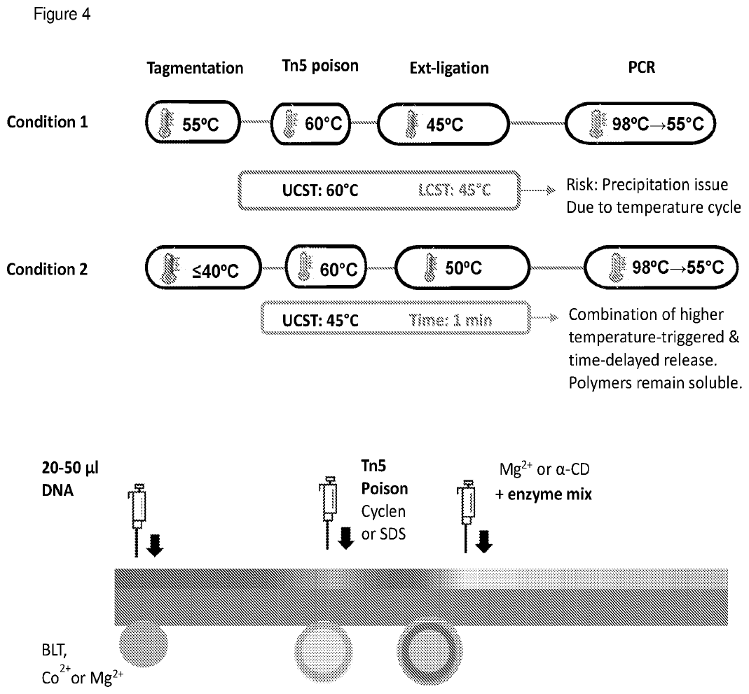

To address this issue, an additional workflow is discussed. Tagmentation is carried out at lower temperature, e.g., less than 42° C. The temperature is then raised to 60° C. to 65° C. for the tagmentation poisoning reaction and first shell release from a UCST polymer having a temperature release condition of approximately 45° C. The temperature is then lowered to approximately 50° C. for about 1 min to facilitate the second shell release condition, which is a UCST polymer and time delay condition, which releases the extension-ligation and PCR reagents into the mixture. The higher temperature for the second release condition minimizes the risk of UCST precipitation since most UCST polymers are soluble above 45° C. In an alternate workflow, the poisoning step is carried out at 60 to 65° C. with a UCST trigger condition set to this time or temperature, and wherein the second shell release is also a UCST with a 45° C. release condition and the reagent is released as the temperature ramps up to the desired temperature. Optionally the second shell also comprises a time delay polymer to allow the poisoning reaction to run to completion and the temperature to cool back down to 50° C. prior to release of the second core extension-ligation and PCR reagents (see illustration in FIG. 4).

Improvement of the lyophilization of reagents may be carried out by minimizing harmful or interfering agents in which active ingredients may be stored. For example, bead linked transposons are stored in glycerol buffer and the tagmentation reaction mixture may include 5% dimethylformamide (DMF). However, glycerol can interfere with the lyophilization process and so may DMF. To minimize the effect of glycerol, BLTs not in glycerol buffer are mixed with cobalt chloride without DMF for the tagmentation reagent mixture. Additionally, the tagmentation reaction temperature is lowered to between 37° C. and 41° C. to provide even sequencing coverage as well as facilitate certain triggered release conditions.

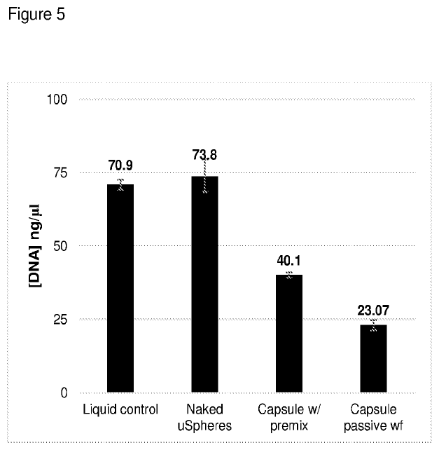

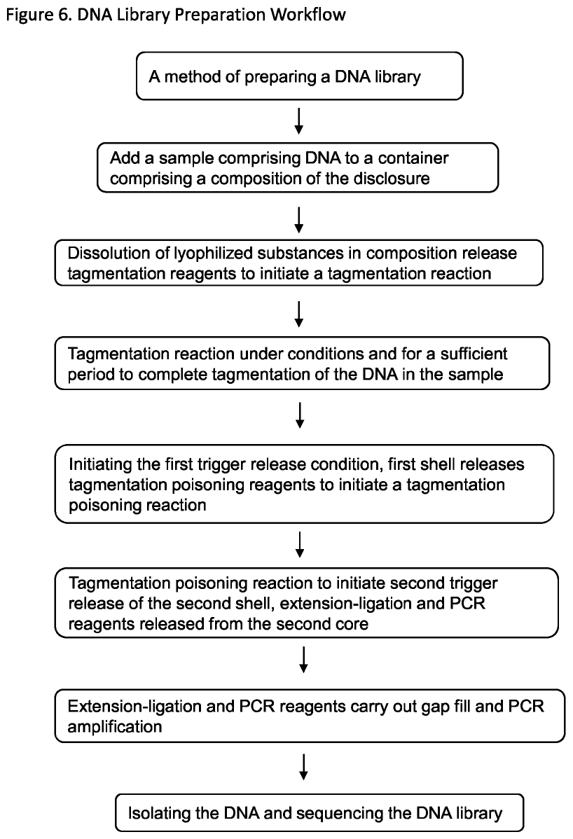

In an alternative formulation, wax (e.g., paraffin wax) is used to encapsulate magnesium-containing microspheres and demonstrate the passive offset of the poison. To test the ability to offset the poison, tagmented DNA (post Tn5 removal) is added to a PCR mix lacking magnesium (Mg). To this mix, either liquid Mg, Mg microspheres or Mg microsphere encapsulated in wax are added. The initial step of the PCR cycling conditions is a 68° C. step which is above the melting temperature of the wax. Large volumes of wax in this format increases the viscosity and decreases yield, and it is hypothesized that encapsulating with UCSTs at the single microsphere level will reduce the amount of material and associated increase in viscosity (FIG. 5).

In an alternative CoCl₂-based workflow, a pH increase is observed between the tagmentation to Tn5 removal processes. This pH increase can range from about pH 6 to about pH 8.5. This natural pH increase is then utilized as a trigger release of the extension-ligation and PCR reagents from microspheres having a pH-dependent polymer shell as described herein. Additionally, it is provided that in a pH-dependent release system, the second microsphere releases a buffer that alters the pH of the reaction. The alteration of pH triggers the dissolution of the shell e.g., a third shell, on a different microsphere in the reaction mixture. This also provides improved pH readiness for the subsequent PCR reaction.

A benefit of using the lyophilized microspheres in a one pot workflow system is that the reagents themselves do not require as much liquid volume, leaving more volume in the preparation tube for sample. Table 4 below shows how much reagent can be used when lyophilized microspheres are used instead of liquid reagent.

TABLE 4
Possible Reagent in Reaction

| [Conc] | Tag Buffer | Poison | Gap-fill/PCR |
|---|---|---|---|
| 2 x | 26 μSpheres | 26 μSpheres | 35 μSpheres |
| | (2.5 mg) | (2.5 mg) | (3.4 mg) |
| 5 x | 11 μSpheres | 11 μSpheres | 14 μSpheres |
| | (1 mg) | (1 mg) | (1.4 mg) |
| 10 x | 5 μSpheres | 5 Spheres | 7 μSpheres |
| | (0.5 mg) | (0.5 mg) | (0.7 mg) |

Numerous modifications and variations of the invention as set forth in the above illustrative examples are expected to occur to those skilled in the art. Consequently, only such limitations as appear in the appended claims should be placed on the invention.

What is claimed is:

1. A composition comprising:
a lyophilized substance comprising one or more tagmentation reagents,
a first particle comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and
a second particle comprising a second shell surrounding a second core, wherein said second shell comprises a hydrophilic or hydrophobic polymer and has a second trigger release condition, and wherein said second core comprises one or more third lyophilized microspheres comprising one or more reagents for extension-ligation and PCR.

2. A composition comprising:
a lyophilized substance comprising one or more tagmentation reagents, and
a first particle comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and
a second shell within the first core and surrounding a second core, wherein said second shell comprises a hydrophilic or hydrophobic polymer and has a second trigger release condition, and wherein said second core comprises one or more third lyophilized microspheres comprising one or more reagents for extension-ligation and PCR.

3. A composition comprising:
a first lyophilized substance comprising one or more tagmentation reagents, and
a first particle comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a second particle comprising a second shell surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

4. A composition comprising:
a first lyophilized substance comprising one or more tagmentation reagents, and
a first particle comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents, and a first outer shell surrounding the first inner shell, wherein the first outer shell comprises a hydrophilic polymer and said first outer shell has a second trigger release mechanism; and
a second shell within the first core and surrounding a second core, wherein said second shell comprises a hydrophilic or hydrophobic polymer and has a third trigger release condition, and wherein said second core comprises one or more third lyophilized microspheres comprising one or more reagents for extension-ligation and PCR.

5. The composition of any one of the preceding claims, wherein the lyophilized substance is selected from the group consisting of lyophilized microspheres, lyophilized beads and a lyophilized cake.

6. The composition of any one of the preceding claims, wherein the first trigger release condition and second trigger release condition are different.

7. The composition of any one of the preceding claims, wherein the trigger release condition is a time release condition, a temperature release condition or a pH release condition.

8. The composition of any one of the preceding claims, wherein the one or more tagmentation reagents is a bead linked transposomes (BLT), transposase, primers, buffers, divalent cations, Tris buffer, cobalt buffer, and/or a lyophilization reagent.

9. The composition of claim 8, wherein the tagmentation reagents comprise bead linked transposomes, primer tags, cobalt chloride and trehalose.

10. The composition of claim 8, wherein the tagmentation reagents comprise bead linked transposomes, magnesium sulfate, Tris buffer and trehalose.

11. The composition of any one of the preceding claims, wherein the one or more tagmentation poisoning reagents is a chelator, deoxynucleotide triphosphates, Tris buffer, potassium buffer, ammonium salts, and/or a lyophilization reagent.

12. The composition of claim 11, wherein tagmentation poisoning reagents comprise Tris buffer, potassium chloride, ammonium sulfate, cyclen tetrahydrochloride, alpha cyclodextrin, dNTPs and trehalose.

13. The composition of claim 11, wherein the tagmentation poisoning reagents comprise potassium chloride, ammonium sulfate, SDS, dNTPs and trehalose.

14. The composition of any one of the preceding claims, wherein the extension-ligation and PCR reagents comprise magnesium sulfate, one or more polymerases, ampligase, NAD₊, betaine, a surfactant and/or a lyophilization reagent.

15. The composition of claim 14, wherein the extension-ligation and PCR reagents comprise magnesium sulfate, glycerol free hot start Q5, tTaq 608 polymerase, ampligase, NAD₊, betaine, Tween-20 and trehalose.

16. The composition of claim 14, wherein the extension-ligation and PCR reagents comprise glycerol free hot start Q5, tTaq 608 polymerase, ampligase, NAD₊, betaine, alpha cyclodextrin, Tween-20 and trehalose.

17. The composition of any one of the preceding claims, wherein the polymer is a water soluble or hydrophilic polymer.

18. The composition of claim 17, wherein the hydrophilic polymer is selected from the group consisting of carboxymethyl cellulose (CMC), carboxymethyl ethylcellulose (CMEC), hydroxyethyl cellulose (HEC), hydroxypropyl cellulose (HPC), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC), pullulan, polyvinyl pyrrolidone, polyvinyl alcohol, copolymers of vinyl pyrrolidone or vinyl alcohol, polyacrylamide, polyacrylic acid, copolymers of ethacrylic acid or methacrylic acid, acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride, or combinations thereof.

19. The composition of any one of the preceding claims, wherein the polymer is a water insoluble or hydrophobic polymer.

20. The composition of claim 19, wherein the hydrophobic polymer is selected from the group consisting of ethyl cellulose (EC), methylethyl cellulose (MEC), cellulose acetate (CA), cellulose acetate phthalate (CAP), cellulose acetate trimellitate (CAT), polyvinyl acetate, fatty acid, fatty acid esters, or combinations thereof.

21. The composition of any one of the preceding claims, wherein the first or second shell comprises an anti-static agent, an anti-foaming agent, an anti-tacking agent, a plasticizer, one or more UCST polymers, one or more LCST polymers, hydroxypropyl methylcellulose, or combinations thereof.

22. The composition of any one of the preceding claims, wherein the first shell comprises Efka 6783, Makon 17R4, polyethylene glycol (PEG) stearate, and a UCST polymer 1.

23. The composition of any one of the preceding claims, wherein the second shell comprises Efka 6783, Makon 17R4, polyethylene glycol (PEG) stearate, a UCST polymer 2 and hydroxypropyl methylcellulose (HPMC).

24. The composition of any one of the preceding claims, wherein the first release condition is a temperature release condition.

25. The composition of claim 24, wherein the temperature for the first release condition is above about 45° C.

26. The composition of any one of the preceding claims, wherein the second release condition is a time release condition or a pH dependent condition.

27. The composition of claim 26, wherein the pH-triggered release condition is a pH increase between about pH 6 to about pH 8.5.

28. The composition of any one of the preceding claims, wherein the composition is stable when stored between about 4° C. to about 37° C.

29. A system for preparing a DNA library comprising one or more containers comprising a composition of any one of claims 1 to 28.

30. A container, comprising
a first lyophilized substance comprising one or more tagmentation reagents,
a first particle comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents, and
a second particle comprising a second shell surrounding a second core, wherein said second shell comprises a hydrophilic or hydrophobic polymer and has a second trigger release condition, and wherein said second core comprises one or more third lyophilized microspheres comprising one or more extension-ligation and PCR reagents.

31. A container comprising,
a first lyophilized substance comprising one or more tagmentation reagents, and
a first particle comprising a first shell surrounding a first core, wherein said first shell comprises a hydrophilic or hydrophobic polymer and has a first trigger release condition, and wherein said first core comprises one or more second lyophilized microspheres comprising one or more tagmentation poisoning reagents.

32. A container, comprising
a first lyophilized substance comprising one or more tagmentation reagents,
a first particle comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents and a first outer shell surrounding the first inner shell, wherein the first outer shell comprises a hydrophilic polymer and said first outer shell has a second trigger release mechanism, and
a second particle comprising a second shell surrounding a second core, wherein said second shell comprises a hydrophilic or hydrophobic polymer and has a third trigger release condition, and wherein said second core comprises one or more third lyophilized microspheres comprising one or more extension-ligation and PCR reagents.

33. A container comprising,
a first lyophilized substance comprising one or more tagmentation reagents, and
a first particle comprising a first inner shell surrounding a first core, wherein said first inner shell comprises a hydrophilic or hydrophobic polymer and has a first trigger release condition, and wherein said first core comprises one or more second lyophilized microspheres comprising one or more tagmentation poisoning reagents and a first outer shell surrounding the first inner shell, wherein the first outer shell comprises a hydrophilic polymer and said first outer shell has a second trigger release mechanism.

34. The container of any one of claims 30 to 33, wherein the one or more tagmentation reagents is a bead linked transposomes (BLT), transposase, primers, buffers, divalent cations, Tris buffer, cobalt buffer, and/or a lyophilization reagent.

35. The container of claim 33, wherein the tagmentation reagents comprise bead linked transposons, primer tags, cobalt chloride and trehalose.

36. The container of claim 33, wherein the tagmentation reagents comprise bead linked transposomes, magnesium sulfate, Tris buffer and trehalose.

37. The container of any one of claims 30 to 36, wherein the one or more tagmentation poisoning reagents is a chelator, deoxynucleotide triphosphates, Tris buffer, potassium buffer, ammonium salts, and/or a lyophilization reagent.

38. The container of claim 37, wherein the tagmentation poisoning reagents comprise Tris buffer, potassium chloride, ammonium sulfate, cyclen tetrahydrochloride, alpha cyclodextrin, dNTPs and trehalose.

39. The container of claim 37, wherein the tagmentation poisoning reagents comprise potassium chloride, ammonium sulfate, SDS, dNTPs and trehalose.

40. A container comprising,
a second particle comprising a second shell surrounding a second core, wherein said second shell comprises a hydrophilic or hydrophobic polymer and has a second trigger release condition, and wherein said second core comprises one or more third lyophilized microspheres comprising one or more extension-ligation and PCR reagents.

41. The container of any one of the claims 30 to 40, wherein the extension-ligation and PCR reagents comprise magnesium sulfate, one or more polymerases, ampligase, NAD₊, betaine, a surfactant and/or a lyophilization reagent.

42. The container of claim 41, wherein the extension-ligation and PCR reagents comprise magnesium sulfate, glycerol free hot start Q5, tTaq 608 polymerase, ampligase, NAD₊, betaine, Tween-20 and trehalose.

43. The container of claim 41, wherein the extension-ligation and PCR reagents comprise glycerol free hot start Q5, tTaq 608 polymerase, ampligase, NAD₊, betaine, alpha cyclodextrin, Tween-20 and trehalose.

44. The container of any one of the claims 30 to 43, wherein the first or second shell comprises an anti-static agent, an anti-foaming agent, an anti-tacking agent, a plasticizer, one or more UCST polymers, one or more LCST polymers, hydroxypropyl methylcellulose, or combinations thereof, or combinations thereof.

45. The container of any one of claims 30 to 39 and 41 to 44, wherein the first shell comprises Efka 6783, Makon 17R4, polyethylene glycol (PEG) stearate, and a UCST polymer 1.

46. The container of any one of claims 30 to 45, wherein the second shell comprises Efka 6783, Makon 17R4, polyethylene glycol (PEG) stearate, a UCST polymer 2 and hydroxypropyl methylcellulose (HPMC).

47. The container of any one of claims 30 to 39 and 41 to 46, wherein the first release condition is a temperature release condition.

48. The container of claim 47, wherein the temperature for the first release condition is above about 45° C.

49. The container of any one of claims 30 to 49, wherein the second release condition is a time release condition or a pH-triggered release condition.

50. The container of claim 49, wherein the pH-triggered release condition is a pH increase between about pH 6 to about pH 8.5.

51. The container of any one of claims 30 to 50, wherein the lyophilized substance, microsphere or particle is stable when stored between about 4° C. to about 37° C.

52. The container of any one of claims 30 to 51, wherein the lyophilized substance is a lyophilized microsphere, lyophilized bead or lyophilized cake.

53. The container of any one of claims 31 to 52, wherein the container is a PCR tube, vial, microtube, flow cell, multiwell plate, glass tube, cartridge or microfluidic chip.

54. A kit comprising a composition of any one of claims 1 to 28 or a container of any one of claims 30 to 53, and instructions for use.

55. A kit comprising a container of claim 31 and a container of claim 40, and instructions for use.

56. A method of preparing a DNA library comprising:
adding a sample comprising DNA to a container comprising a composition of any one of claims 1 to 28, wherein addition of the sample initiates dissolution of the first lyophilized substances to release the one or more tagmentation reagents into the container to initiate a tagmentation reaction;
allowing the tagmentation reaction to continue under conditions and for a sufficient period to complete tagmentation of the DNA in the sample;
initiating the first trigger release condition, wherein the first trigger release condition of the first shell releases the one or more tagmentation poisoning reagents of the first core into the container to initiate a tagmentation poisoning reaction; and
allowing the tagmentation poisoning reaction to continue under conditions and for a sufficient period to initiate the second trigger release condition of the second shell, wherein the one or more extension-ligation and PCR reagents are released from the second core, wherein the extension-ligation and PCR reagents carry out gap fill and PCR amplification.

57. A method of preparing a DNA library comprising:
adding a sample comprising DNA to a container of any one of claims 30 to 53, wherein addition of the sample initiates dissolution of the first lyophilized substances to release one or more tagmentation reagents into the container to initiate a tagmentation reaction;
allowing the tagmentation reaction to continue under conditions and for a sufficient period to complete tagmentation of the DNA in the sample;
initiating the first trigger release condition, wherein the first trigger release condition of the first shell releases one or more tagmentation poisoning reagents of the first core into the container to initiate a tagmentation poisoning reaction; and
allowing the tagmentation poisoning reaction to continue under conditions and for a sufficient period to initiate the second trigger release condition of the second shell, wherein the one or more extension-ligation and PCR reagents are released from the second core, wherein the extension-ligation and PCR reagents carry out gap fill and PCR amplification.

58. The method of claim 56 or 57, further comprising isolating the DNA and sequencing the DNA library.

59. The method of any one of claims 56 to 58, wherein the tagmentation reaction is carried out between about 37° C. to about 41° C. for about 5 minutes to about 15 minutes.

60. The method of any one of claims 56 to 59, wherein the first release condition is a temperature release condition.

61. The method of claim 60, wherein the temperature for the first temperature release condition is above about 45° C.

62. The method of any one of claims 56 to 61, wherein the tagmentation poisoning reaction is carried out at between about 60° C. to about 65° C. for about 1 minute to about 5 minutes.

63. The method of any one of claims 56 to 62, wherein the second release condition is a time release condition or a pH-triggered release condition.

64. The method of claim 63, wherein the time release condition is from about 1 minute to about 3 minutes at about 50° C.

65. The method of claim 63, wherein the pH-triggered release condition is a pH increase between about pH 6 to about pH 8.5.

66. The method of any one of claims 56 to 65, wherein the one or more extension-ligation and PCR reagents are released from the second core at about 50° C., wherein the extension-ligation and PCR reagents carry out extension, ligation and gap fill.
